(19) United States
(12) Patent Application Publication
(10) Pub. No.: US 2021/0085467 A1
(43) Pub. Date: Mar. 25, 2021

(54) SELECTIVE TARGETED RELEASE

(71) Applicant: WEST VIRGINIA UNIVERSITY, Morgantown, WV (US)

(72) Inventors: Matthew J. DIETZ, Morgantown, WV (US); Graig A. MARX, New Kensington, PA (US)

(73) Assignee: WEST VIRGINIA UNIVERSITY

(21) Appl. No.: 17/100,151

(22) Filed: Nov. 20, 2020

Related U.S. Application Data
(63) Continuation-in-part of application No. PCT/US2019/033577, filed on May 22, 2019.
(60) Provisional application No. 62/675,024, filed on May 22, 2018.

(51) Int. Cl. A61F 2/30; A61F 2/38

(52) U.S. Cl. CPC A61F 2/3094; A61F 2/38; A61F 2002/30985; A61F 2002/30156; A61F 2002/30113; A61F 2002/30154; A61F 2002/30143; A61F 2002/3068

(57) ABSTRACT

A spacer system replaces or substitutes for a medical implant in a patient. The system includes a housing comprising a shape similar to the medical implant; an aperture in the housing, a subcutaneous port configured to receive a medicament, the subcutaneous port being in fluid communication with the aperture; and at least one channel within the housing, wherein the at least one channel extends from the aperture to an exterior surface of the housing to equally distribute a material the medicament from the aperture through the at least one channel. The subcutaneous port is in fluid communication with the aperture through a catheter connecting the subcutaneous port to the aperture. The system may be used to replace a variety of medical implants, including a hip implant, an intramedullary nail, a pedicle screw, a knee implant, a sternum prosthesis, and a clavicle prosthesis, an ankle implant, shoulder implant, tibia implant, femur implant, humerus implant, spinal cage, external fixation pin, intercalary fusion device, talus implant, or a vertebral body implant.

### CROSS-REFERENCE TO RELATED APPLICATIONS

This is a Continuation-In-Part of International Patent Application No. PCT/US2019/033577, filed May 22, 2019, published as International Patent Publication No. WO 2019/226805. The entire disclosure of the prior application is hereby incorporated by reference herein in its entirety.

### TECHNICAL FIELD

Various embodiments disclosed herein relate generally to devices for providing selective targeted treatment release.

### BACKGROUND

The number of patients requiring joint replacement or internal fixation devices is steadily increasing. The global market for hip and knee orthopedic surgical implants was valued at $17 billion in 2015 and is projected to grow to $33 billion by 2022. Osteoarthrosis is the major indication for joint replacement surgery. It mostly affects middle-aged and elderly patients. As such, demand for joint replacement surgery is driven by a growing geriatric population across the world. Due to an increasingly active population, rising obesity demographics, and a broadening patient base including younger patients, there is an increased lifetime risk of complications.

The use of biomaterials in orthopedic surgery involves a high risk of developing infections. Bacterial infection of joint prosthesis is one of the most challenging complications in orthopedics. As the patient population with implant associated infection increases, the incidence of patients with such infections continues to increase. One study reported revision rates of about 6% after five years and 12% after ten years.

Evidence indicates that prosthesis infections are biofilm-correlated infections that are highly resistant to antibiotic treatment and the host immune response. Since intravenous (IV) antibiotics alone are not capable of eradicating biofilm infections, a curative therapy usually includes surgery. The traditional procedure is a two-stage exchange of the implant. The first stage includes removal of the implant and placement of an antibiotic spacer. After surgery, patients typically need at least 6 weeks of intravenous (IV) antibiotics. Once the infection has been cured, patients undergo revision surgery (stage 2). During this procedure, the antibiotic spacer is removed, and a new implant is placed.

An antibiotic spacer is a device placed into the joint to maintain joint space and alignment while an infection is being treated with IV antibiotics. Spacers are typically made of polymethyl methacrylate (PMMA), commonly known as bone cement, that is loaded with antibiotics. The positioning of antibiotic-loaded cement into the surgical site is useful to maintain a high concentration of drug at a local level, reducing the risk of systemic toxicity. However, bone cement was not designed as an antibiotic delivery agent. Despite the passage of almost four decades since it was first used, there are still some limitations in the use of antibiotic-loaded bone cement, including: the method of preparation, the choice of antibiotic, the effective release and diffusion of the antibiotic in the surrounding tissue, and the mechanical properties of loaded cement.

The method of preparation of the spacers is one of the most important factors that affect the release of antibiotics and the mechanical properties of PMMA. The process of polymerization of bone cement is an exothermic reaction with temperatures up to 60-80 degrees Celsius. Therefore, antibiotics destined to be mixed with PMMA must be chemically and thermally stable. In addition, the antibiotic mixed with bone cement must have a broad antibacterial spectrum and a low percentage of resistant species. High doses of antibiotics may play a role in weakening the structure and the mechanical properties of PMMA.

Once placed in the infection site, release of the antibiotic from the spacer is influenced by the viscosity of bone cement, the surface of the contact/exchange, the conditions of the compound, and the type and amount of antibiotic. Most of the antibiotic is released in the first hours and days after surgery while significant amounts may remain trapped in the cement for a long time. The concentration of antibiotics released can rise above the minimum inhibitory concentration (MIC) which is capable of treating free floating, planktonic bacteria. However, to treat implant associated infections levels 100-1000× higher are required known as the minimum biofilm eradication concentration (MBEC). Current delivery agents are unable to achieve these concentrations. Systemic release and risk of toxicity prevents loading current spacer with enough antibiotics to achieve this level.

Successful therapy treats prosthesis infection for a long-term pain-free and functional joint. However, successful eradication of implant associated infection using two-stage exchange is severely limited by the antibiotic spacer. The failure rate of the two-stage exchange can be as high as 25-30%. There is a need in the art for the ability to adjust the type and dosage of antibiotic and other anti-infection treatments delivered to treat prosthesis infections more efficiently and effectively.

### SUMMARY OF EXEMPLARY EMBODIMENTS

A brief summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a spacer including a housing, an aperture, and at least one channel or conduit within the housing. Connected to the aperture is tubing that leads to a subcutaneous port. The port is accessible from external the body with a needle. The port can be placed near or remote from the housing as desired. The at least one channel extends from the aperture to an exterior surface of the housing to equally distribute a material from the aperture through the channels to the desired location(s). The material may be a treatment agent that may be equally distributed to an infection site of a patient, such as an intra-articular, intramedullary, cancellous bone or soft tissue site of a patient.

Various embodiments additionally relate to a spacer that includes at least one channel that extends to an exterior surface of the housing at a particular location chosen to treat an infection.

Various embodiments additionally relate to the spacer including a subcutaneous port that is subcutaneous when implanted.

Various embodiments relate to a spacer that can include a reservoir.

Various embodiments relate to a spacer including a channel that includes an oval, circular, triangular, square, irregular, or hexagonal cross-section.

Various embodiments relate to a method of manufacturing a spacer by printing a solvent-soluble channel pattern for use in a PMMA spacer mold. The solvent-soluble channel pattern may be a water-soluble channel pattern, or an organic solvent-soluble channel pattern.

Various embodiments relate to a method of manufacturing a spacer which is the same shape as an implant replaced by the spacer including at least one channel extending from an aperture through the spacer to an exterior surface of said housing.

Various embodiments disclosed herein relate to a spacer to replace a medical implant in a patient, including a housing comprising a shape similar to the medical implant; an aperture, a subcutaneous port; and at least one channel within the housing. The at least one channel extends from the aperture to an exterior surface of the housing to equally distribute a material from the aperture through the channels. In various embodiments, the medical implant is a knee implant, hip implant, ankle implant, shoulder implant, intramedullary nail (femur, tibia, humerus, external fixation device), pedicle screw or cage; and the spacer may or may not include a reservoir within the housing. The at least one channel may extend to an exterior surface of the housing at a particular location chosen to treat an infection, tumor, or bone loss. In various embodiments, the subcutaneous port remains within the patient but outside the affected area. In various embodiments, the subcutaneous port and the spacer may be placed in a patient to administer medication to a patient with an infection, such as a bacterial biofilm, in tissue surrounding an implant, or on the surface of an implant. In some embodiments, the subcutaneous port and the spacer may be used to replace an implant in a patient with a tumor or other cancer near the site of the implant and used to administer chemotherapeutic agents to the patient. If bone loss near an implant occurs, the implant may be replaced with a spacer, which is used to administer bone stimulating or osteoclast inhibiting bone augmentation agents to the surrounding tissue. In some embodiments, a spacer may be positioned in a patient in place of a conventional implant, where the patient is at high risk due to prior infections, tumors, or bone loss. Should such a situation reoccur, a subcutaneous port may be implanted and connected to the spacer to allow drug administration.

Various embodiments disclosed herein relate to a spacer to replace a medical implant in a patient, including a housing comprising a shape similar to the medical implant; an aperture; and at least one channel within the housing, wherein the at least one channel comprises an oval, circular, triangular, or hexagonal cross-section. The at least one channel may equally distribute a material to an infection site of a patient, wherein the infection site is cancellous bone or a soft tissue site. The material may be a treatment agent, such as an antibiotic, antifungal, bacteriophage fluid, antimicrobial peptides, a surfactant, an enzyme, a wash, an antiseptic, pain medication, a chemotherapeutic agent, an immunotherapeutic agent, an anti-thrombotic agent, and an anti-inflammatory.

Various embodiments disclosed herein relate to a spacer to replace a medical implant in a patient, including a housing comprising a shape similar to the medical implant; an aperture; and at least one channel within the housing, wherein the at least one channel has a constant or variable diameter of between 100 to 2000 microns.

Various embodiments disclosed herein relate to a method of manufacturing a spacer by printing a solvent-soluble channel pattern for use in a pre-existing spacer mold, wherein the water-soluble channel pattern may be printed using additive manufacturing techniques, e.g., 3D printing.

A method of manufacturing a spacer may include manufacturing a spacer to replace a medical implant, by printing the spacer using an additive manufacturing technique, e.g., 3D printing, wherein the spacer has the same shape as the medical implant replaced by the spacer.

Additive manufacturing may be performed using a variety of polymeric and/or metallic materials, by methods known in the art. Suitable materials include polymethylmethacrylate; nylons and other polyamides, other plastics, e.g., polyolefins or polyesters; cobalt chrome; stainless steel; and/or titanium. Spacers prepared by additive manufacturing may be designed for temporary, semipermanent, or permanent implantation. In various embodiments, the spacers 100 may be designed without an internal reservoir; the external reservoir port 62 is used as a reservoir for distribution of drug to channels 120 in spacer 100. In some embodiments, the spacers 100 may include a supplemental reservoir for distribution of drug received from port 62.

If spacer 100 will be left in the patient for an extended period of time, channels 120 and/or openings 121 in spacer 100 may be treated with an antithrombotic agent, e.g., a solution of heparin in glycerin. If desired, the antithrombotic agent may be replenished by injecting an antithrombotic agent into port 62 in between steps of drug administration into the spacer 100 through port 62.

Various embodiments disclosed herein relate to a preform for a spacer to replace a medical implant in a patient, the preform including a housing comprising a shape similar to the medical implant; an aperture; and a 3D-printed solvent-soluble network pattern within the housing, wherein the 3D-printed network pattern extends from the aperture to a plurality of openings on an exterior surface of the housing. The solvent-soluble network pattern is configured to be dissolved from the housing so as to form channels in the medical implant, wherein the channels place the aperture in fluid communication with the plurality of openings. The housing has a shape similar to a medical implant, where the medical implant may be a hip implant, an intramedullary nail, a pedicle screw, a knee implant, a sternum prosthesis, or a clavicle prosthesis.

Various embodiments disclosed herein relate to a spacer to replace a medical implant in a patient, including a housing with a shape similar to the medical implant; an aperture; and a network of channels within the housing. The network of channels extends from the aperture to a plurality of openings on an exterior surface of the housing. The spacer is prepared from a preform including a housing comprising a shape similar to the medical implant; an aperture; and a 3D-printed solvent-soluble network pattern within the housing. The method includes steps of dissolving the 3D-printed water-soluble network pattern from the housing of the preform so as to form the network of channels, where the network of channels places the aperture in fluid communication with the plurality of openings.

Various embodiments disclosed herein relate to a method for replacing a medical implant in a patient, where the implant is at a site in need of treatment, by removing the medical implant from the patient, and replacing the medical implant with a spacer comprising a housing comprising a shape similar to the medical implant; an aperture; and at least one channel within the housing, wherein the at least one channel extends from the aperture to an exterior surface of the housing. A subcutaneous port configured to receive a medicament may be implanted in the patient, and placed in fluid communication with the aperture through a catheter connecting the subcutaneous port to the aperture. The method may further include a step of supplying the medicament to the subcutaneous port in an amount sufficient to cause the medicament to travel through the catheter to the at least one channel in the housing, and a step of supplying an anticoagulant to the subcutaneous port in an amount sufficient to cause the anticoagulant to coat the at least one channel in the housing. In various embodiments, the site in need of treatment is a site of infection, a site undergoing bone loss, or a site of a tumor.

### BRIEF DESCRIPTION OF THE DRAWINGS

In order to better understand various exemplary embodiments, reference is made to the accompanying drawings, wherein:

FIG. 1 illustrates a known method of treating a patient with an implant associated infection;

FIG. 2 illustrates antibiotic elution using a conventional spacer;

FIG. 3 illustrates a method of treating an infection in a patient near an implant according to the present disclosure;

FIG. 4 illustrates administration of antibiotics using an STTR spacer;

FIGS. 5A-5B illustrate a perspective and side view, respectively, of an embodiment of the spacer having channels and a connection for a subcutaneous port;

FIG. 5C illustrates a view of an embodiment of the spacer of FIG. 5A in combination with a subcutaneous port;

FIG. 5D illustrates a subcutaneous port implanted under the skin;

FIG. 6A illustrates an in vivo spacer connected to an aperture and a tube for administration of a fluid;

FIG. 6B illustrates an embodiment of a spacer having a fused port and tube for administration of a fluid;

FIG. 6C illustrates an explanted spacer with an aperture connected a tube for administration of a fluid, where clotting has occurred at the spacer surface;

FIG. 7 illustrates a top side view of an embodiment of the spacer having network channels;

FIGS. 8A-8B illustrate proximal and distal angular views, respectively, of an embodiment of the knee spacer having loop channels;

FIGS. 9A and 9C illustrate distal and proximal views, respectively, of the knee spacer having loop channels;

FIG. 9B illustrates a distal perspective view of the knee spacer having loop channels;

FIG. 10A illustrates a distal view of the knee spacer having a cistern channel system;

FIG. 10B illustrates a top view of the knee spacer having a cistern channel system;

FIG. 11A illustrates a cross-sectional view of an embodiment of a spacer having a hexagonal channel;

FIG. 11B illustrates a cross-sectional side view of a channel having a changing diameter;

FIG. 12A illustrates a side view of an embodiment of a spacer having channels for use in the hip;

FIG. 12B illustrates an embodiment of a preform for a set of channels for use in the hip spacer, manufactured using an additive manufacturing method using water-soluble materials;

FIG. 12C illustrates a cross section of an embodiment of a preform for hip spacer, including the hip spacer formed around a soluble preform for a set of channels;

FIG. 12D illustrates a cross section of an embodiment of a preform for hip spacer, formed from the preform of FIG. 12C by dissolving the soluble preform for the set of channels;

FIG. 12E illustrates an embodiment of a preform for hip spacer with a network of channels, formed from the preform of FIG. 12C by dissolving the soluble preform for the set of channels;

FIG. 13A to 13F illustrate various 3-D printed knee spacers;

FIGS. 14A and 14B illustrate a spacer implanted in a cadaver specimen;

FIG. 15 illustrates the even distribution of fluid through a spacer with channels;

FIGS. 16A-16D illustrate a pedicle screw spacer having channels for the administration of fluid;

FIG. 17 illustrates an in-vitro comparison of elution from an embodiment of the spacer as compared to Palacos and Simplex PMMA spacers;

FIGS. 18A to 18C illustrate implantation of a knee spacer and a subcutaneous port in a sheep;

FIGS. 19A-19B illustrate a comparison of synovial fluid vancomycin concentration from an embodiment of the spacer as compared to a Palacos Cement Spacer;

FIGS. 19C-19D illustrate synovial fluid vancomycin concentration from an embodiment of the STTR spacer (FIG. 19B) as compared to a Palacos Cement Spacer (FIG. 19A), where the channels are treated with an anticoagulant formulation;

FIGS. 20A-20B illustrate a comparison of quantifiable serum concentrations of vancomycin from an embodiment of the STTR spacer (FIG. 20B) as compared to a Palacos Cement Spacer (FIG. 20A);

FIGS. 21A-21C illustrate implantation of a knee spacer in a cadaver, and administration of drug to the surrounding tissue;

FIGS. 22A-22B illustrate implantation of a hip spacer in a cadaver, and administration of drug to the surrounding tissue;

FIGS. 23A-23B illustrate implantation of pedicle screw spacers in a cadaver, and administration of drug to the surrounding tissue; and

FIGS. 24A-24F show various embodiments of an aperture for an embodiment of the spacer having channels.

### DETAILED DESCRIPTION OF THE INVENTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art, and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

An antibiotic-loaded cement spacer is fundamental in two-stage revision surgery. The spacer helps maintain a grade of joint stability, add an intra-articular and/or intramedullary concentration of antibiotics, and keep the patient's anatomical area of any foreign infected prosthetic material during infection eradication.

Typically, the antibiotic-loaded cement spacer is static or dynamic. Static spacers keep the joint in full extension or minimal flexion. Static spacers commonly result in joint stiffness with a poor range of motion after the second stage of the revision. Furthermore, static spacers may not restore the normal anatomic joint contours, particularly in heavier patients, leading to significant bone loss with a higher risk for spacer displacement.

Dynamic antibiotic-loaded cement spacers allow flexion and extension of the joint between the two surgical stages. Allowing motion during the surgical stages is useful in preventing scar tissue formation around the joint. As such, revision surgery in patients with a dynamic spacer may be easier than in those with a static one. Moreover, the patient's ability to bend the joint increases quality of life between stages, especially if a long period of antibiotic therapy is necessary to eradicate the infection. However, instability and wound healing problems have been associated with the use of dynamic spacers. The use of commercially available dynamic spacers is also limited by the availability of implant sizes. Dynamic spacers are also more expensive than static spacers.

Static and dynamic spacers are further limited by the type and dose of antibiotic mixed into the bone cement. In each case, it is essential to know which bacteria are responsible for the infection. However, even when bacteria are reported as sensitive to an antibiotic, they may not respond to the same antibiotic when growing in biofilm. Thus, at least 6 weeks of systemic IV antibiotic therapy is administered in conjunction with the antibiotic spacer. The bacteria may not be determined or cultured until after a spacer and antibiotic are in place. If the bacteria are found to be resistant to the antibiotics placed in the spacer, the spacer may no longer be effective if a culture later determines resistance to the antibiotic used in the spacer.

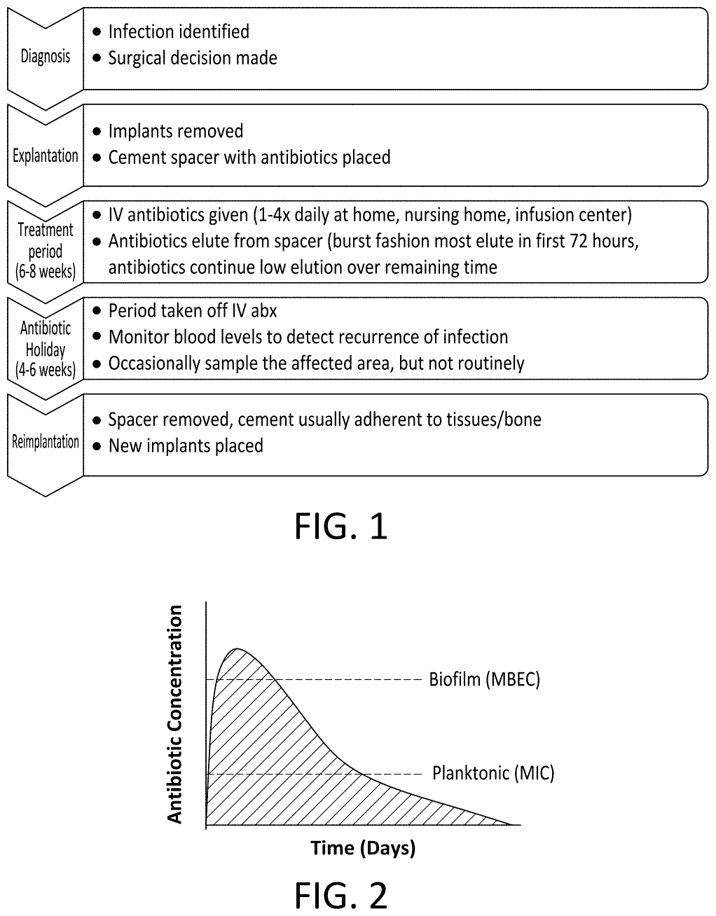

Known procedures are outlined in FIG. 1. Once an infection is identified, a decision to surgically remove the implant may be made (Diagnosis). When the implant is removed, a cement spacer infused with antibiotics may be inserted (Explantation). The antibiotics elute from the spacer in a burst mode for about 72 hours, and then the remaining antibiotics may elute gradually. Since most antibiotics elute from the cement spacer within 72 hours, intravenous antibiotics must be administered for 4 to 6 weeks after explantation (Treatment Period). At the end of this time, intravenous antibiotics are stopped (Antibiotic Holiday). If infection does not recur during a 6 to 8 week monitoring period, the spacer is removed and a new implant is inserted (Reimplantation).

There are certain disadvantages with the procedure shown in FIG. 1. There is a limited antibiotic selection of suitable antibiotics which may be present in the spacer. There are limited options for modifying dosage levels. Should an infection be caused by an organism which is resistant to the antibiotic within the spacer, the antibiotic in the spacer cannot readily be changed without further surgical intervention to remove the spacer and replace it with a new spacer infused with a different drug. During the Treatment Period when intravenous antibiotics are administered, only a portion of the antibiotics are delivered to the affected site. Delivery of intravenous antibiotics systemically in amounts suitable to treat the infection may cause toxicity. Since the bulk of the antibiotics in the spacer are eluted within about 72 hours, the spacer produces antibiotic levels which are below the minimum biofilm eradication concentration (MBEC) throughout the treatment period. While drug levels and/or pathogen levels in the blood can be monitored during the treatment period, it is difficult to directly monitor drug levels or the presence of an infection at the site of the spacer. The long course of treatment increases the likelihood that drug resistance will develop. A Peripherally Inserted Central Catheter (PICC Line) extending outside the body is required; however, the PICC line may be a source of additional infection and blood clots. Finally, the procedure of FIG. 1 offers no ability to augment treatment to improve bone quality at the end of the treatment period.

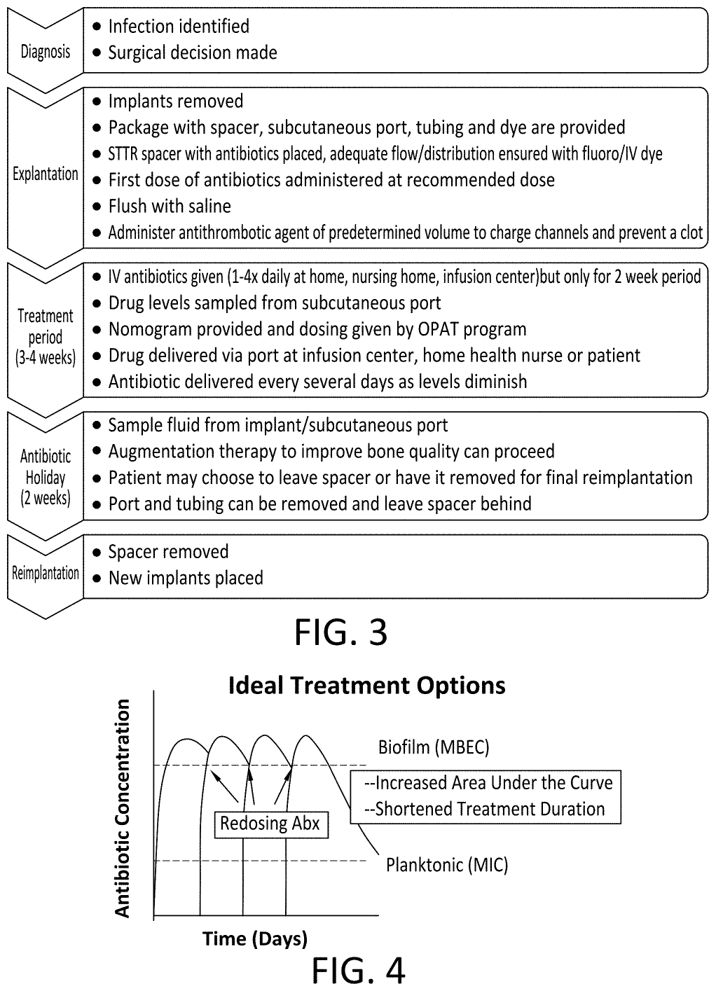

FIG. 2 shows antibiotic levels achieved during the Treatment Period by direct elution from the spacer in the procedure of FIG. 1. During an initial burst period, the spacer produces antibiotic levels which are above the minimum biofilm eradication concentration (MBEC), and may act to control an infection present as a biofilm near the spacer. However, within a short period of time, the spacer produces antibiotic levels which are below the MBEC, and will not control infections caused by pathogens which form biofilms. The spacer instead produces antibiotic levels which are above the minimum inhibitory concentration (MIC) for planktonic (free-living) organisms, but insufficient to eradicate biofilms.

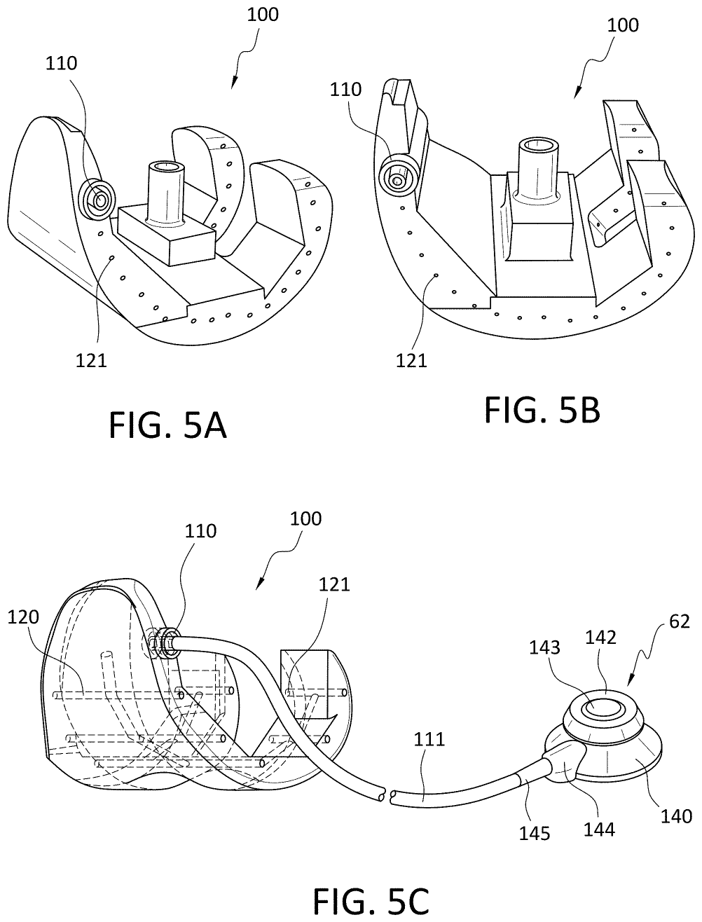

FIG. 3 shows a procedure according to the present disclosure. Once an infection is identified, a decision to surgically remove the implant may be made (Diagnosis). When the implant is removed, a selective targeted treatment release (STTR) spacer may be inserted (Explantation). The spacer may be part of a package with an STTR spacer, a subcutaneous port, and tubing connecting the port to the spacer. The spacer may include an aperture to engage the tubing, and a network of passages or channels within the spacer which connect the aperture to an external surface of the spacer. Once the STTR spacer is positioned, a radio-opaque or fluorescent dye may be injected into the subcutaneous port, and the patient may be monitored to insure adequate flow/distribution of the dye from the subcutaneous port through the tubing to the network of passages within the spacer. Once adequate flow is confirmed, a first dose of antibiotics may be administered to the STTR spacer at a recommended dose. Optionally, the system may then be flushed with saline, and an antithrombotic agent of predetermined volume may be administered to charge the passages within the spacer and prevent clot formation.

The Treatment Period of FIG. 3 is shown as lasting from 3 to 4 weeks. However, depending on the needs of the patient, the Treatment Period may last from 1 to 12 weeks, from 1 to 8 weeks, from 10 days to six weeks, from 12 days to five weeks, from two weeks to four weeks, or from three to four weeks, and involves IV antibiotics given periodically, e.g., at intervals ranging from two hours to one week, from four hours to four days, from six hours to two days, from 8 hours to one day, or from twelve hours to one day. IV antibiotics may be given at home, at a nursing home, at an infusion center, or a doctor's office or outpatient clinic for a two week period. Drug levels near the spacer may be sampled from the subcutaneous port. An antibiotic or other drug may be delivered through the subcutaneous port directly to the tissue around the spacer at an infusion center or outpatient clinic, or by a home health nurse or by the patient. Antibiotics are delivered to the spacer periodically as levels diminish, so as to maintain a drug level at the spacer which exceeds the MBEC. FIG. 3 shows administration of antibiotics 1 to 4 times daily. However, depending on the needs of the patient, antibiotics may be administered at intervals ranging from about two hours to about seven days, from six hours to five days, from twelve hours to four days, or from one to three days. During an Antibiotic Holiday lasting from 1 week to six weeks, from 10 days to four weeks, from 12 days to three weeks, from two weeks to three weeks, or about two weeks, as shown in FIG. 3, fluid from the implant is sampled through the subcutaneous port to ensure that reinfection does not occur. Augmentation therapy to improve bone quality may proceed. The patient may choose to leave the spacer in place, or have it removed for final reimplantation. The subcutaneous port and tubing may be removed, while leaving the spacer in place. Alternatively, the spacer may be removed and a new implant may be inserted (Reimplantation). Depending on the needs of the individual patient, the length of the Treatment Period and Antibiotic Holiday described above may be shortened or increased as needed. Additionally, antibiotics or other drugs may be administered at intervals which may be adjusted to meet the needs of the individual patient.

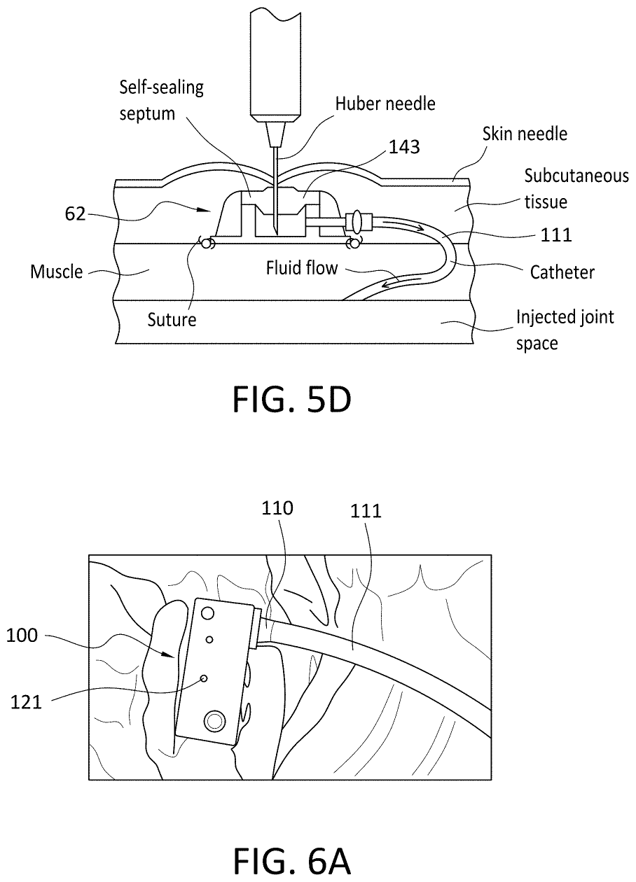

FIG. 4 shows antibiotic levels achieved during the Treatment Period obtained by the procedure of FIG. 3. After an initial infusion of antibiotics from the subcutaneous port through the spacer, the STTR spacer produces antibiotic levels which are above the minimum biofilm eradication concentration (MBEC), and may act to control an infection present as a biofilm near the spacer. However, within a short period of time, the antibiotic levels begin to decline. At this point, additional doses of antibiotics may be infused into the STTR spacer, so that the antibiotic levels in the tissue are maintained above the MBEC. This has the advantage of providing an increased Area Under the Curve (AUC), and a shortened antibiotic treatment duration.

The selective targeted treatment release (STTR) spacer of the disclosure provides highly controlled and targeted delivery of treatment agents to the infection site of a patient. In various embodiments, the treatment agents may include antibiotics, surfactants, enzymes, washes, antiseptics, anti-inflammatories, or other treatments of infectious disease and/or inflammation that are known in the art. In various embodiments, the infection site may be an intra-articular, intramedullary, cancellous bone or soft tissue location of the patient.

The procedure shown in FIGS. 3 and 4 allows direct delivery of drugs/washes to affected environment. The affected environment may be a joint with an artificial implant, including a knee, hip, ankle, or shoulder joint. The joint may include a bone with an artificial implant, where the bone may be the tibia, including the proximal tibia; femur, including a total femur replacement; humerus; talus; or spine. Spinal implants may include pedicle screws, bone plates, or vertebral bodies. The affected environment may be fixed in position with external fixation pins. The affected environment may include a fusion device, e.g., for intercalary fusion of bones in a joint.

The procedure shown in FIGS. 3 and 4 may be used to address a variety of issues in the local environment surrounding the spacer, including infections in or near the bone, including osteomyelitis. Such infections may include prosthetic joint infections. Metal implants may be created for 1.5-stage exchange arthroplasty, allowing replacement of an articulating implant with a spacer, with the intent for the spacer to remain in place for a prolonged time. Non-metal implants may be created for 2-stage exchange arthroplasty, allowing replacement of an articulating or static implant with an articulating or static spacer. The articulating or static spacer may then be removed after the infection has been successfully treated, allowing reimplantation with a new implant.

The procedure shown in FIGS. 3 and 4 may also be used to address bone nonunion, e.g., the inability to repair a fracture. An implant may be used to connect bone fragments. The procedure may also be used to address tumors in a bone or joint, as well as metabolic abnormalities causing bone resorption. A spacer may also be used in place of a conventional implant when treating a high-risk patient with a prior history of joint infections, tumors, or significant bone loss.

The procedure shown in FIG. 3 may be used to administer a variety of drugs to tissue surrounding an infected or otherwise damaged joint or bone through the spacer. Such drugs can include antimicrobials, antibiotics, antifungals, bacteriophage solutions, antimicrobial peptides, anti-inflammatories, bone stimulating or osteoclast inhibiting bone augmentation agents, pain medication, chemotherapeutic agents, antithrombotic agents, or mixtures thereof. In between drug administration steps, the channels or passages in the spacer may be washed with saline, or with an additional bioactive agent, such as a biofilm eradicating agent, a bone building solution, or a culture enabling wash. Additionally, biological agents may be administered to the joint through the spacer. Such biological agents include cells harvested from the patient, stem cells, bone stimulating agents, and/or growth hormones.

The spacers disclosed herein allow administration of drugs to infected joints with improved concentrations. Drug levels achieved with the device can achieve minimum biofilm eradication concentration (MBEC) quickly, where the MBEC is usually 100-1000 times the minimum inhibitory concentration (MIC) of free infectious organisms. The spacers allow rapid administration of drugs to achieve detectable levels within the bone and blood, with high local levels of drug within the affected joint or tissue. Non-toxic drug levels are achieved systemically, despite high local levels. As the local drug levels decline below MBEC, the spacer may be redosed to increase local drug levels.

In contrast, current techniques only achieve a minimum inhibitory concentration of a drug, and are not maintained at a steady state during a six week course of treatment. Current spacers provide drug levels in burst elution, with an initial level exceeding MBEC, but then rapidly declining to the MIC. Current spacers are made of a cement infused with drug, and cannot be readily reinfused with a therapeutic agent. The spacers disclosed herein can be redosed, and drug levels at the MBEC can be maintained for a longer period of time.

The STTR spacer allows the injection of treatment agent(s) via an aperture, into channels included inside the spacer that allow the treatment agent or agents to flow evenly out into the infected joint and surrounding tissues. These channels may be positioned to maximize the effectiveness and efficiency of treatment by delivering higher doses of a treatment for an infection for an initially longer period, adjusting the type of antibiotic, antimicrobial, or other treatment delivered, and controlling the concentration of infection treatment delivered. Treatments may include all fluid treatments known in the art and include, but are not limited to, antibiotics, antimicrobials, sulfate washes, and enzymes.

In various embodiments, the spacer further allows the injection of non-treatment agents, such as an antithrombotic agent infused to prevent clogging of the channels. Suitable antithrombotic agents include heparin-related agents, or tPa agents, such as alteplase.

In various embodiments, the channels may be positioned such that flow to surrounding tissues is equally distributed. The channels may be spaced within the spacer in various shapes. The channels may extend from a central channel line connected to an aperture. Various embodiments further include channels extending from a cistern. Various embodiments include a channel which loops around the spacer having channels extending therefrom to the exterior of the spacer. The channels may have various diameters to provide equal distribution of fluid delivered therethrough. Suitable diameters include from 100 to 2000 microns, preferably from 500 to 2000 microns, more preferably from 1000 to 2000 microns. In various embodiments, the diameter may be adjusted dependent upon how much fluid is to be distributed or to provide equal distribution among multiple channels. In various embodiments, a single channel may change in diameter to adjust fluid flow.

In various embodiments, the channels may be characterized by various cross-sectional shapes. The channels may have any shape, including, but not limited to circular, oval, triangular, and hexagonal. Circular and oval shapes are less likely to occlude and may be preferable in spinal spacers. Hexagonal channels may provide improved strength and may be preferable in knee spacers. In various embodiments, each channel may include one or more cross-sectional shapes.

In various embodiments, the size and location of the channels may be customized to ensure efficient and effective delivery of infection treatments. The channels may vary in location based on an individual patient or the type of spacer. The infection location may be specifically targeted in a STTR spacer customized for an individual patient. In various embodiments, channels may be distributed equally around the spacer. Various spacers may also include channels that are directed to a particular location or locations of the spacer. Various channels may lead to an articular surface of the spacer. Various embodiments include spacers having channels evenly distributed around the spacer.

In various embodiments, the STTR spacer is manufactured to the dimensions of implants available in the art. In further various embodiments, the STTR spacer may be designed from patient scan data, implant dimensions, etc. The shape and size of the spacer may be customizable to best fit each patient's needs. The resulting spacer is similar (if not identical) to the implant that is removed, allowing for better mobility while treating the infection. The fluid injection channel locations and diameters may also be customized to ensure efficient and effective delivery of infection treatments to the infected area. The spacer shape and similarity to the implant to be removed results in a spacer that is partially weight-bearing, provides improved motion, and reduces potential bone loss due to ratcheting of bone cement on bone.

In various embodiments, the STTR spacer may be manufactured using additive manufacturing methods, such as 3D printing and the like. In various embodiments, designs may be printed using any suitable material, including but not limited to PMMA, nylon, acrylic derivative resins, other resins, stainless steel, cobalt chromium, and titanium. Methods used for spacer production may include, but are not limited to, fused deposition modeling, stereolithography, and selective laser sintering. In alternative embodiments, channels may also be manufactured using additive manufacturing methods using solvent-soluble materials. The channels may then be used in existing molds for manufacturing spacers. The solvent-soluble material may then be dissolved and removed from a finished spacer.

The STTR spacer allows for continuous and dynamic delivery of treatments for infection to a targeted area of infection. Because the treatment is not mixed with the bone cement, physicians have more flexibility in choosing an adequate type and dose of treatment to treat the infection. Furthermore, physicians may be able to change the therapy if they encounter antibiotic resistance. Thus, the need for IV antibiotics may be reduced, resulting in less frequent treatment, shorter treatment times and lower costs. Moreover, the STTR spacer design is completely customizable to the needs of each patient, which reduces instability while allowing full range of motion and results in improved quality of life during treatment.

The STTR spacer may be used in various joint procedures, including but not limited to knee and hip surgeries. The STTR spacer may further be used as a: shoulder spacer prosthesis; ankle replacement prosthesis; intramedullary femoral, tibial or humeral nail; plate for osteosynthesis; a drug delivery device for nonunion treatment; and spinal instrumentation, such as a fusion rod and interbody spacer or pedicle screw in the treatment of discitis. In various embodiments, the STTR spacer may include an articular recess to allow for better or customized articulations.

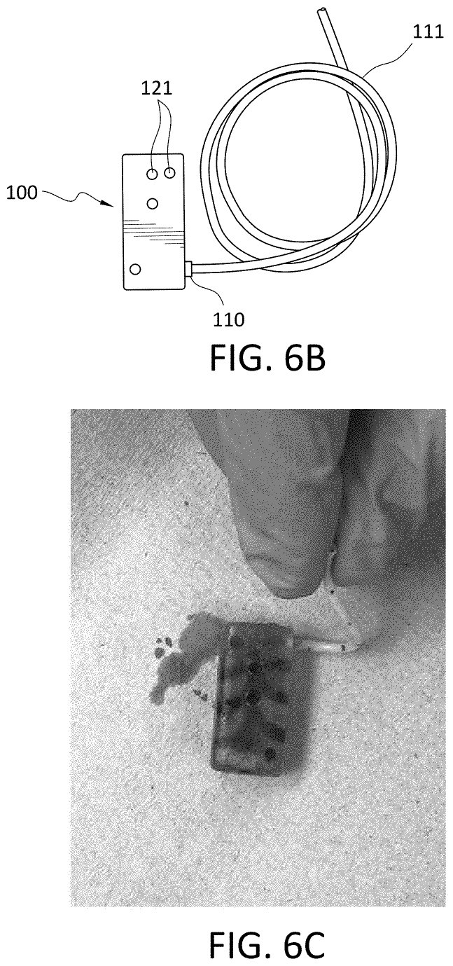

FIGS. 5A and 5B illustrate a side and perspective view of an embodiment of the spacer 100, configured for use in the knee. The spacer has an aperture connection 110 through which fluid infection treatments may be administered to the patient. The fluids travel through channels 120 (shown in FIGS. 2-4) in the spacer and exit through openings 121 on the surface of the spacer. In various embodiments, the aperture connection 110 may be subcutaneous. As shown in FIGS. 5A and 5B, the aperture connection 110 may be a Luer lock type connection or press fit. The aperture connection 110 may allow for removal of a catheter without the removal of the spacer from a patient. FIG. 5C shows a spacer 100 in combination with a subcutaneous port 62. The subcutaneous port 62 includes a self-sealing septum 143, a rim 142 surrounding the septum, a base 140, and a hollow reservoir within the base below septum 143. An exit from the reservoir through parts 144 and 145 places the interior of the reservoir in fluid communication with the interior of a flexible tube or catheter 111. The interior of tube or catheter 111 is in fluid communication with channels 120 of spacer 100 through aperture 110. A drug or other agent may be injected into subcutaneous port 62 through septum 143, and will then pass through tube or catheter 111 into channels 120 of spacer 100. The agent then enters tissue surrounding the spacer 100 through openings 121 in the surface of the spacer.

As shown in FIG. 5D, the subcutaneous port 62 is implanted under the skin line, and sutured to the underlying muscle. A syringe with a hypodermic needle, e.g., a Huber needle, may be used to inject a therapeutic agent through septum 143 into a reservoir. The therapeutic agent then travels through tube or catheter 111 into an infected joint space.

The subcutaneous port 62 may also be used to assess the condition of the local tissue by sampling the local environment by aspirating fluids or cellular material through the subcutaneous port with a syringe. Such aspiration allows fluids to be assessed for cell count, or cultured for the presence of infectious agents. Tissues and/or fluids aspirated through the subcutaneous port may be tested for the presence of inflammatory biomarkers. Drug concentrations in the infected joint may be assessed as a function of time, allowing determination of when additional doses of a therapeutic agent are required.

Administration of a drug through a system as disclosed herein is done through the subcutaneously contained port 62. Port 62 is implanted so that it is spaced from, but localized near, the environment affected by an infection or other condition. A drug is injected into the reservoir within port 62. The reservoir in port 62 is in fluid communication with channels 120 in spacer 100 through tube or catheter 111, and through channels 120 with openings 121 in the surface of spacer 100. Thus, a drug can be administered into tissue surrounding spacer 100 by injecting the drug into port 62, remote from the spacer. Treatment with a drug therefore does not require repeated entry into the infected tissue or environment by a syringe or other instrument. Once the infection has been addressed, the doctor is able to remove the port 62 without requiring a visit to the operating room. Port 62 can be removed with a small incision in an outpatient setting. After removal of port 62, spacer 100 may be replaced with a conventional implant, or left in place in the patient. For patients who have had prior infections or prior incidence of bone loss, medical personnel may elect to leave spacer 100 in place in the event that further treatment is required.

Port 62 and spacer 100 can each be removed from the patient's body, or port 62 can be removed while leaving spacer 100 behind, if desired. The height of port 62 and the length of tube or catheter 111 can be varied as desired. The reservoir in port 72 can be accessed with a small gauge needle, e.g., 21 gauge or 27 gauge. The combination of port 62, spacer 100 and tube or catheter 111 is well tolerated by the patient, and allows for repeated dosing of the drug into the reservoir and spacer, without disruption of the port 62 or leaking of drug from the port 62. A line carrying drugs is not required outside the body, and no continual connection of port 62 to a pump or infusion device is required.

The package provided to medical personnel for administration to the patient includes the spacer or implant 100, the subcutaneous port 62, and the tubing or catheter 111 configured to connect the exit from the reservoir in port 62 to aperture connection 110 in spacer 100. The kit may also include a radio-opaque dye to test patency of the system, where the dye may be injected into port 62 to insure that the dye travels through catheter 111 to spacer 100, and exits through openings 121, without leakage around port 62.

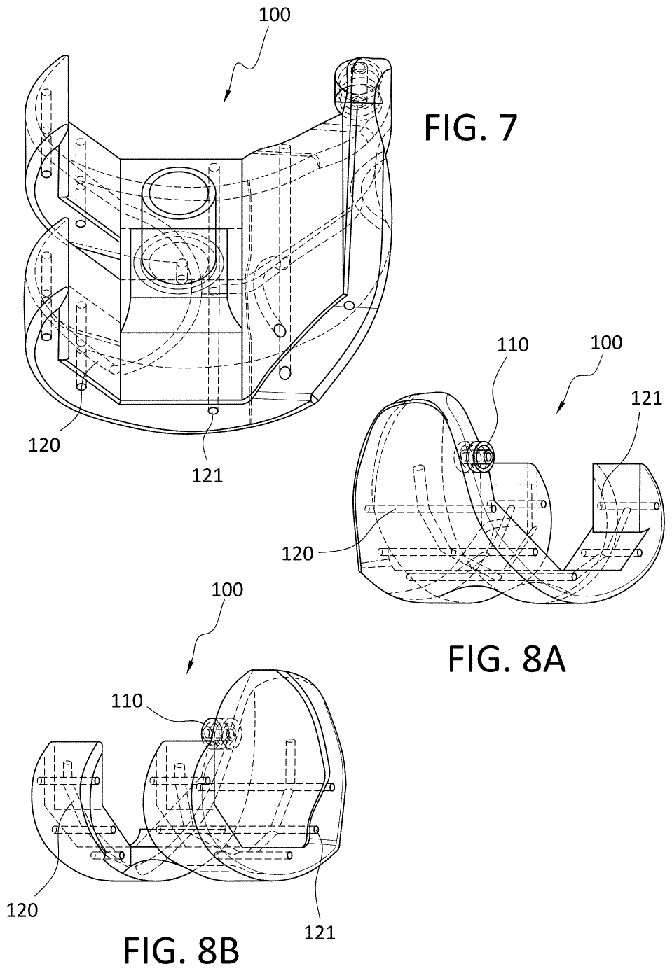

FIG. 6A illustrates an embodiment of a spacer 100 with an aperture 110 connected to tubing 111 for administration of the infection treatments. In alternative embodiments, the spacer 100 may include an aperture 110 and tubing 111 permanently fused together using a polymeric material, as shown in FIG. 6B. In various embodiments, channels 120 and/or openings 121 may be treated with an antithrombotic agent to prevent clotting. FIG. 6C shows a spacer 100 with an aperture 110 connected to tubing 111, where clotting has occurred at channels 120 and/or openings 121 in vivo due to the absence of an antithrombotic agent.

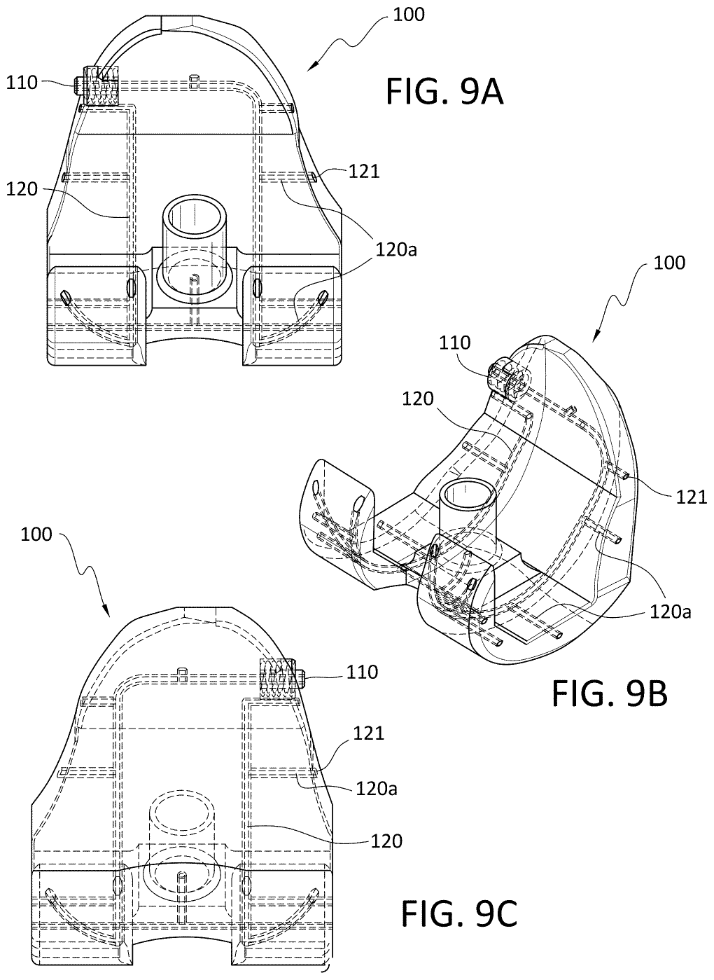

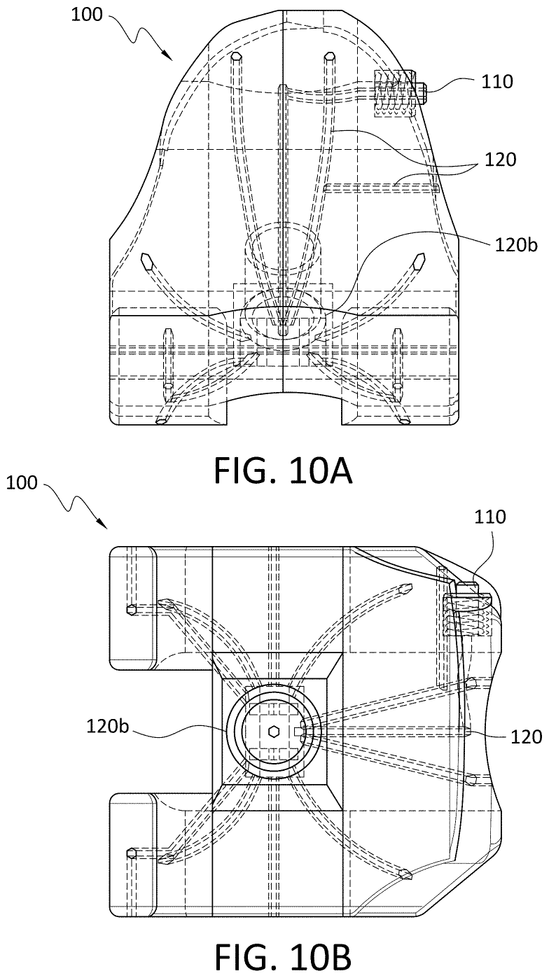

FIGS. 7-10 illustrate paths the channels 120 may form in an embodiment of a spacer 100 for use in the knee. The channel paths may be selected or adjusted to direct fluid to a particular location and to provide equal distribution of the fluid through the spacer. FIGS. 7, 8A, and 8B illustrate a knee spacer having network channels 120. The channels 120 in a network extend from a central line (not shown) connected to an aperture 110. Network channel distribution may or may not include a reservoir for collecting a drug. The fluids are dispersed through the channels 120 to all surfaces of the spacer implant in such a spacer.

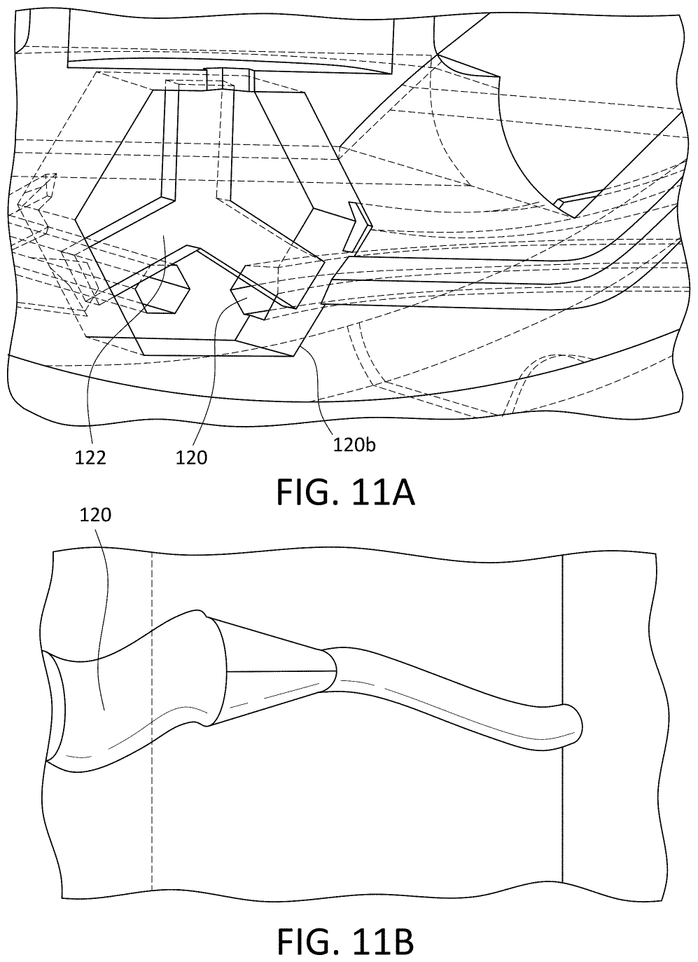

FIGS. 9A-C illustrate a spacer having loop channels. A channel 120 loops around the spacer and additional channels 120a branch from the looping channel and extend to openings 121 at the surface of the spacer. Loop channel distribution may not include a reservoir for collecting a drug.

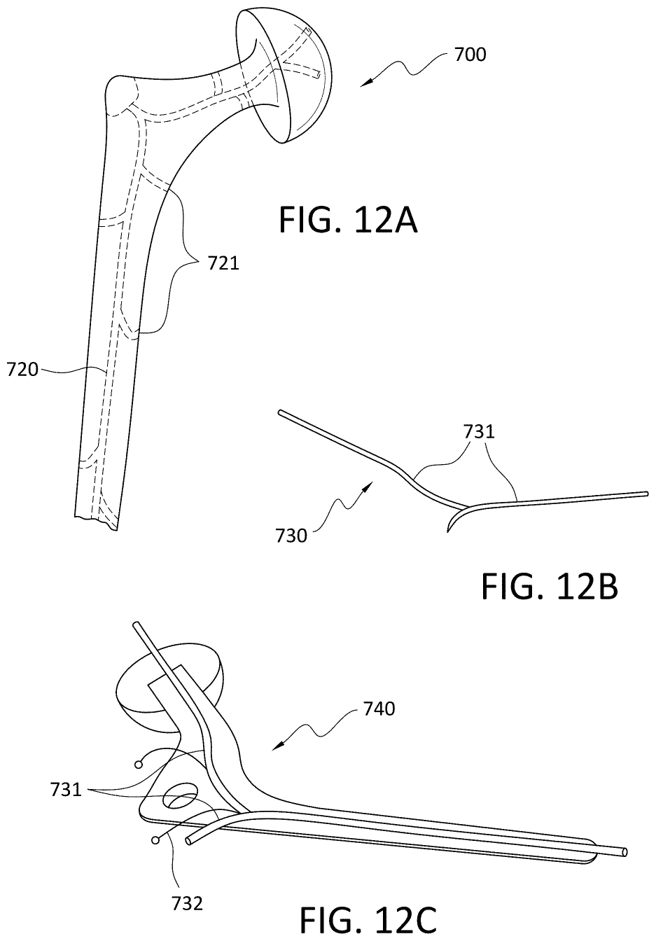

FIGS. 10A and 10B illustrate a spacer 100 having cross-sectional hexagonal shaped channels 120. The channels 120 extend from a cistern channel 120b, i.e., a circular channel, that may act as a hub to allow for the equal active distribution of pharmaceuticals and disperse them equally to all channels. Channels 120 extend in a generally radial manner from cistern channel 120b. A cistern channel system may have a selective channel distribution.

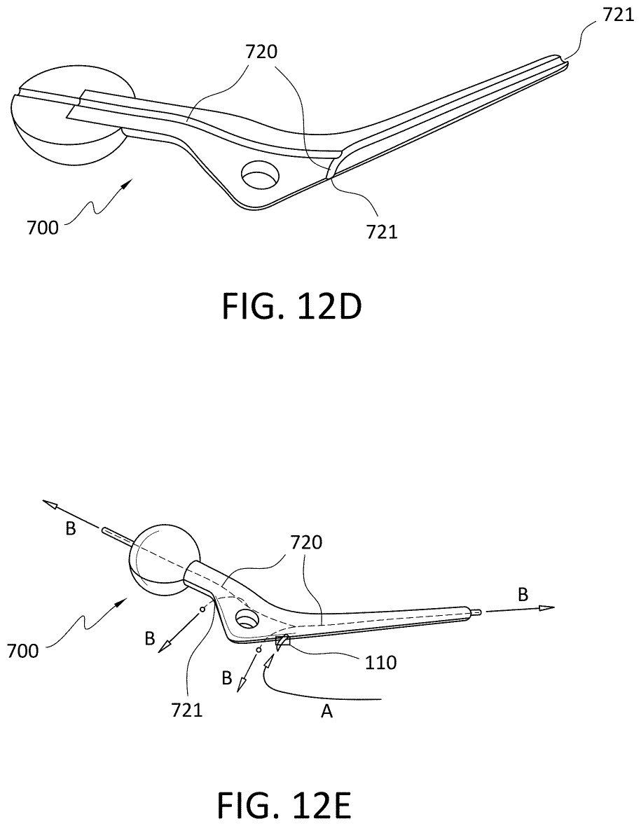

FIG. 11A illustrates a cross-sectional view of a hexagonal spacer channel 120 extending from a cistern channel 120b. FIG. 11A further illustrates internal supports 122 within the cistern channel, which may itself be hexagonal.

FIG. 11B illustrates a side view of a channel 120 having a changing channel diameter. The channel diameter may change through any of the channels 120 of a spacer 100. The change in diameter may affect the rate a fluid is released from a channel. The change in diameter may further provide equal distribution of the fluids through several channels. Channels within a single spacer may have a variety of diameters and changes in diameter.

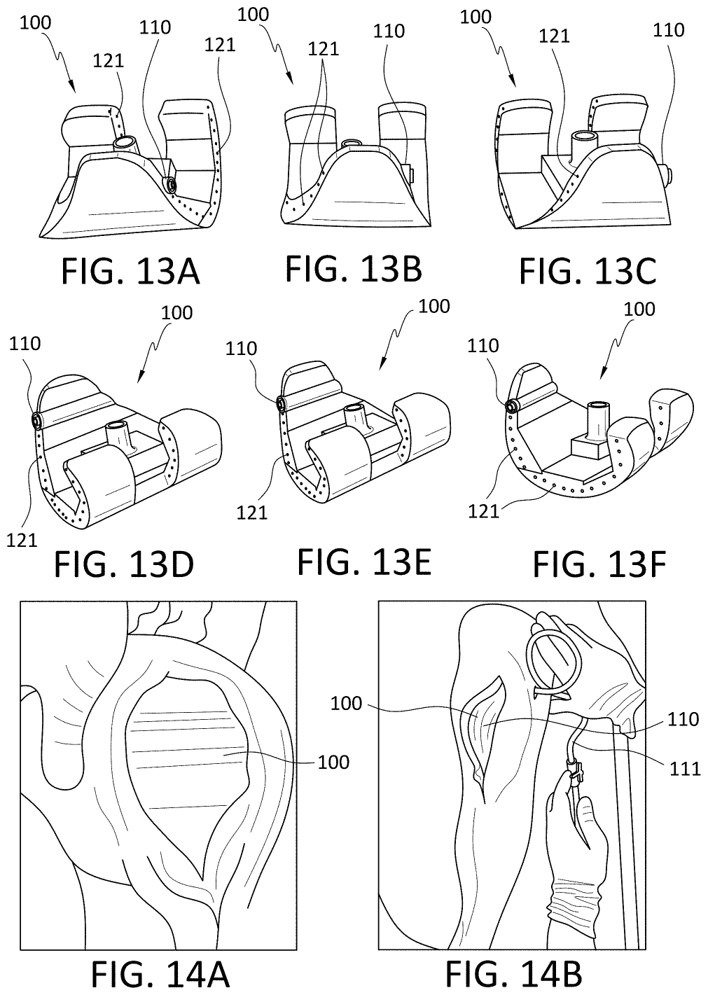

FIG. 12A illustrates an embodiment of a spacer 700 having channels 720 for use in a hip, where channels 720 extend to openings 721 at the surface of spacer 700.

In various embodiments, a spacer, which may be a hip spacer 700, a knee spacer, an intramedullary nail spacer, a pedicle screw spacer, or any other spacer corresponding in shape to a prosthetic bone implant, may be made by designing a 3D network of channels and printing the 3D network from a solid solvent-soluble material. The spacer may then be 3D printed or molded from an insoluble, biocompatible material, where the spacer is formed around the solvent-soluble 3D network. The 3D network extends from an inlet port to an opening, or a plurality of openings, at the surface of the implant. Once the spacer is formed around the solvent-soluble network, the solvent-soluble network is dissolved, forming at least one channel, or a plurality of channels, which place the inlet port in fluid communication with openings at the surface of the spacer. In various embodiments, the solvent-soluble network is soluble in a solvent which may be, but is not limited to, water. The solvent-soluble network may also be made from a polymer which is soluble in an appropriate organic solvent.

After formation, the 3D-printed water-soluble network pattern may be placed into a preexisting mold for formation of a cement spacer, e.g., a polymethylmethacrylate spacer. The spacer may be molded around the 3D-printed water-soluble network pattern. The 3D-printed water-soluble network pattern may then be dissolved from within the spacer to produce a spacer 100 with a network of hollow channels 120 in an operating room. At least one of the channels has an end connected to aperture 100, so as to be connectable to tubing 111 and port 62. Hollow channels 120 place aperture 100 in fluid communication with openings 121 on the surface of spacer 100.

Alternatively, after formation, the 3D-printed water-soluble network pattern may be placed into a 3D printing apparatus, and a water-insoluble spacer may be printed around the 3D-printed water-soluble network pattern. Again, the 3D-printed water-soluble network pattern may be dissolved from within the 3D-printed water-insoluble spacer. In some embodiments, a water-soluble network pattern and a water-insoluble spacer may be printed simultaneously from different resins.

FIG. 12B illustrates a preform 730 for a 3D network of channels, printed from a solid water-soluble material, such as polyvinyl alcohol (PVA) or AirWolf's HydroFill Water-Soluble Support. Preform 730 includes a network of water-soluble solid filaments 731.

In various embodiments, a preform 730 for a 3D network of channels may be printed from a solid organic solvent-soluble material. High impact Polystyrene (HIPS) is a 3D-printable polymer which is soluble in limonene. HIPS may be used in situations where contact with water is unwanted, and HIPS prints 3D structures more easily than PVA. The spacer may then be 3D printed or molded around the network of channels from a water and limonene insoluble, biocompatible material. Limonene may then be used to remove the 3D HIPS network preform, forming a network of open channels 120. Polycaprolactone may be used to print a 3D network of channels, and the spacer may then be 3D printed or molded around the network of channels. A variety of organic solvents, including methylene chloride and toluene, may then be used to remove the 3D network preform, forming a network of open channels 120.

In various embodiments, a preform 730 for a 3D network of channels may be printed with a first set of filaments 731 extending from a port 110, as shown in FIG. 8C (port 110 not shown), where first filaments 731 are printed from a water-soluble resin, e.g., PVA. The preform 730 may be printed with a second set of filaments 732 connected to channels 731. Filaments 732 may be printed from an organic solvent-soluble resin, e.g., HIPS. In some embodiments, filaments 731 are wider than filaments 732.

FIG. 12C illustrates a cross section of a preform 740 for a spacer for use in a hip. Spacer 740 is made of a water insoluble solid material, which may be molded or 3D printed around soluble preform 730 of FIG. 12B. The solid 3D network of first filaments 731 in preform 730 includes a network of water-soluble solid first filaments 731 which pass through preform 740. The network of first filaments 731 extends to the exterior surface of implant preform 740. At least one of first filaments 731 contacts an aperture 110 (not shown in FIG. 12C). In the embodiment of FIG. 12C, first filaments 731 are wide filaments, and first filaments 731 contact second filaments 732. Second filaments 732 may be narrower than first filaments 731, and may extend from filaments 731 to a surface of preform 740.

FIG. 12D illustrates a cross section of a spacer 700 for use in a hip. Spacer 700 is made from preform 740 by dissolving the water soluble solid material from first filaments 731 in preform 740 in FIG. 8C. Dissolving the solid 3D network of first filaments 731 from preform 740 leaves a network of hollow channels 720 which pass through spacer 700. The network of channels 720 extends from an aperture (not shown in FIG. 12D) to openings 721 on the exterior surface of spacer 700, so as to place openings 721 in fluid communication with the aperture. In various embodiments, a spacer 740 as shown in FIG. 12D may be made by designing a spacer with voids corresponding to the desired channels 720, and 3D printing spacer 700 with channels 720 in a single step, without requiring a step of dissolving a solid 3D network.

At least one of first filaments 731 contacts an inlet port 110. In the embodiment of FIG. 8C, the preform 730 for a 3D network of channels includes first filaments 731, which may be wide filaments 731, and second filaments 732 in contact with first filaments 731. At least second filaments 732, and in some embodiments both first filaments 731 and second filaments 732, contact the surface of preform 740. In some embodiments, filaments 731 are printed from a water-soluble resin, e.g., PVA, and filaments 732 are printed from an organic solvent-soluble resin, e.g., HIPS. Filaments 731 may be wider than filaments 732. The water-soluble resin in first filaments 731 may be dissolved selectively to form an open network of wide channels, without affecting filaments 732. If desired, after the water-soluble resin in wide filaments 731 is dissolved, the organic solvent-soluble resin in filaments 732 may be dissolved to form an open network of narrow channels in fluid communication with the wide channels. If desired, filaments 731 may be printed from an organic solvent-soluble resin, and filaments 732 may be printed from a water-soluble resin.

FIG. 12E shows a spacer 700 for use in a hip. Spacer 700 includes a network of channels 720 and an aperture 110. A fluid containing a biologically active material, such as an antibiotic, enters port 110 in the direction of arrow A. The network of channels 720 extends from the aperture 110 to openings 721 on the exterior surface of spacer 700, so as to place openings 721 in fluid communication with the aperture. The fluid flows through the network of channels, which may include channels of varying thickness, to openings 721, and leaves the spacer in the direction of arrows B.

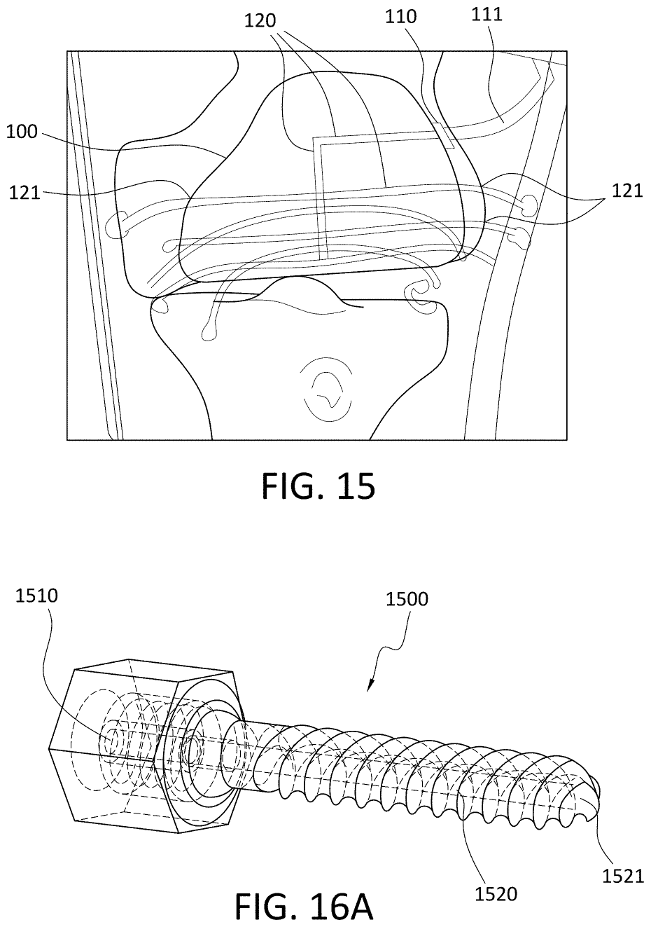

FIGS. 13A to 13F illustrate various 3-D printed knee spacers 100 of the disclosure, each having channels, an aperture 110, and channel openings 121 on the exterior surface of the knee spacers. The aperture 110 is in fluid communication with the channel openings 121 through the channels (not visible in FIGS. 11A to 11F).

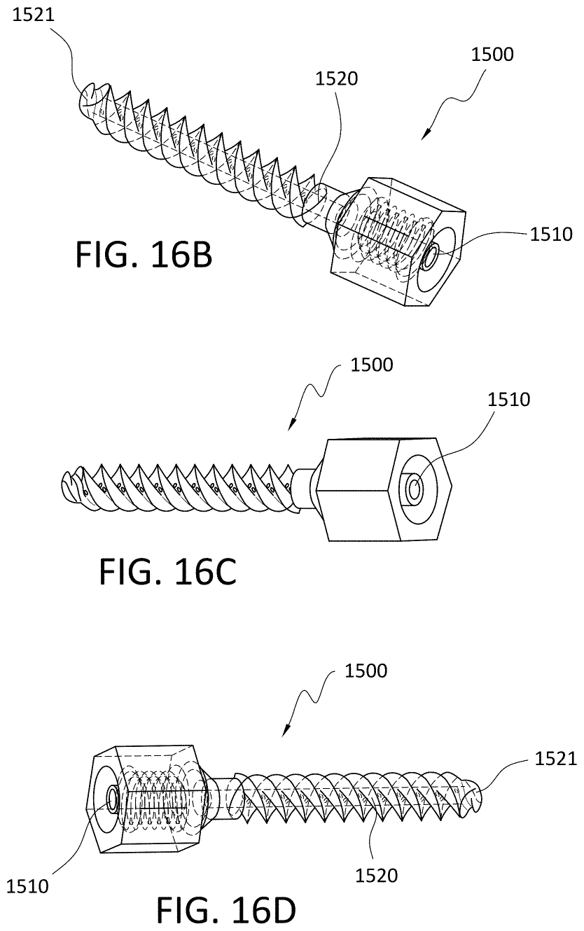

FIGS. 14A and 14B illustrate a knee spacer 100 implanted in a cadaver specimen, where the knee spacer has channels to deliver fluid infection treatments. In FIG. 14B, a fluid material may be delivered through the channels of the spacer. The knee spacer has an aperture 110 in fluid communication with the channels, where the fluid material is delivered to aperture 110 through tubing 111.

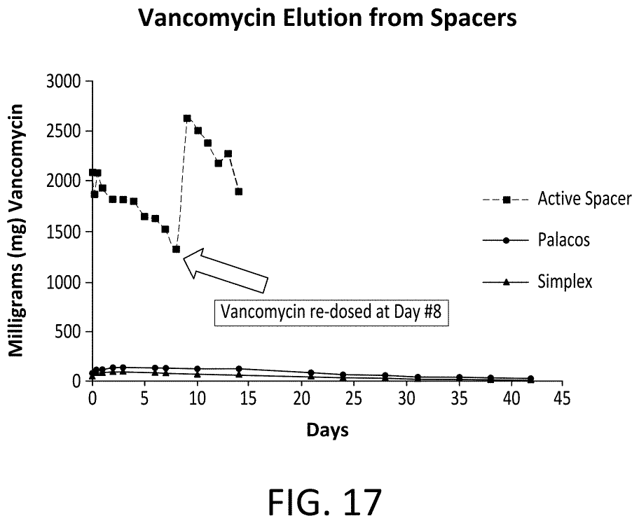

FIG. 15 shows an in-vitro demonstration of the knee spacer on a bone. FIG. 15 illustrates the paths of fluid through a knee spacer 100 manufactured using a Formlabs Form2 SLA 3D printer having channels. The fluid is administered through an aperture 110 through tubing 111, and exits the spacer through channel openings 121 on an exterior surface of the spacer 100 into the joint space in order to effectively treat infections. The fluid is distributed through the spacer 100 from aperture 110 to channel openings 121 through channels 120. The fluid is visualized in the view of FIG. 15 using a dye.

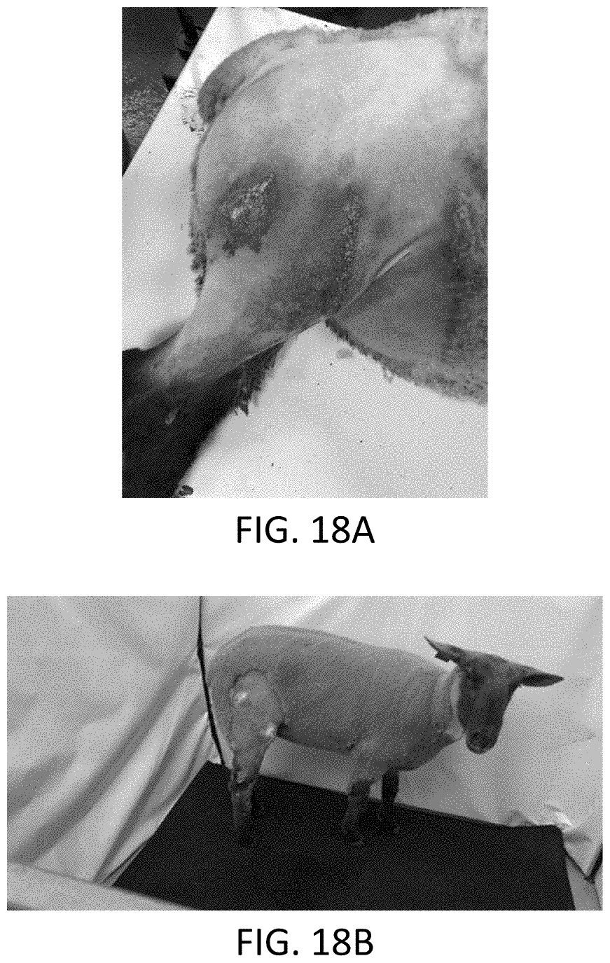

FIGS. 16A-D illustrate a pedicle screw spacer 1500 having channels 1520. Pedicle screw spacers are typically used in the setting of spine surgery, i.e., in the treatment of discitis. When treating discitis, as well as other local spine infections, local infusion methods are limited. In various embodiments, the pedicle screw spacer 1500 includes an aperture 1510 to receive fluid treatment. The fluid is administered through the aperture 1510 and exits the pedicle screw spacer 1500 through a channel opening 1521 on an exterior surface of the pedicle screw spacer 1500 into the vertebral space. Channel opening 1510 is in fluid communication with aperture 1510 through at least one channel 1520.

Some advantages of the present STTR spacers are that they allow improved articulation for joints. The spacers disclosed herein may be used to replace infected or damaged implants, while maintaining motion within the joint and prevent contracture scar tissue during the healing process while the spacer is used to treat infections or other issues. By improving treatment of infections, issues during a second surgery for reimplantation of a conventional implant may be avoided.

The spacers disclosed herein maintain mobility for the patient during treatment, improve patient satisfaction, and shorten hospital stays and/or rehab duration following explantation of an infected implant. The disclosed spacers improve diffusion of the antibiotic from the subcutaneous port through the infected bone or joint.

The spacers disclosed herein also provide improved stability of bones during treatment. In the case of spinal implants, e.g., pedicle screws, spinal stability needed to protect neural structures can be maintained, even after the removal of infected hardware. In the case of other bones, e.g., bones of the arms, legs, knees, or hips, stability of bones is improved. Improved bone healing is obtained using spacers which effectively replace implants, so as to limit the need for external fixation, bracing, or casting. The location of channels in the spacers allows for improved biodistribution of drugs into joints, synovial fluids or cavities, bones, muscles, and connective tissues of the joints. Some benefits of the disclosed spacers include that the administration of drugs to the spacer through the infusion port does not require a hospital setting. The port allows for convenient dosing at home, in an infusion center, in an outpatient clinic, or in a doctor's office. Administration of drugs into the spacer 100 through port 62 allows increased local concentration of a therapeutic agent, e.g., an antibiotic, and allows administration of reduced levels of systemic IV antibiotic, for a reduced period of time. In various embodiments, the spacer may not include an internal reservoir for passive elution of a drug into channels 120, and then into the surrounding tissue. Passive elution may increase the likelihood for potential clotting of blood at or near openings 121 in spacer 100. Active distribution of drug from port 62 to spacer 100 allows clotting to be reduced by administration of an antithrombotic agent in between dosages of an antibiotic or other drug. Also, a patient or nurse can directly feel the subcutaneous port, allowing easy administration of drugs through the port 62 with a syringe.

### EXAMPLES

### Example 1

### STTR Spacer Implant Design and Preparation

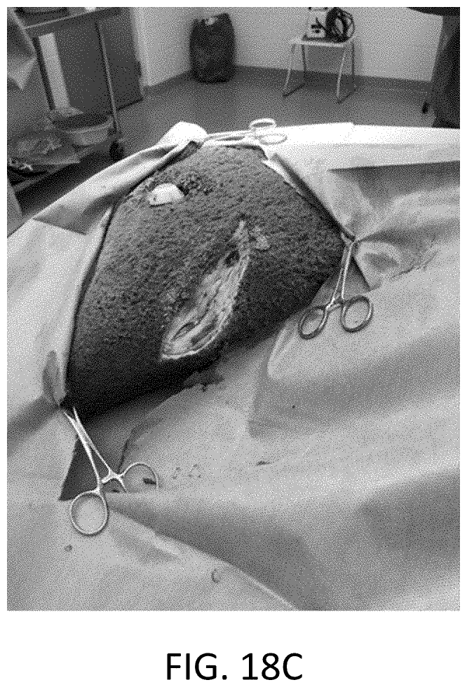

FIG. 17 illustrates the dispersion of an antibiotic administered through various spacers. Antibiotics were readministered in the active spacer and provided more antibiotic distribution over conventional spacers.

A selective targeted treatment release (STTR) spacer was designed with Autodesk Fusion 360 software and printed by additive manufacturing on a Formlabs Form2 SLA 3D printer utilizing a methacrylic acid ester material and a photoinitiator that allows the polymerization to occur using UV Light. The spacer was constructed with a fixed channel diameter of 2.00 mm which then is connected to a Luer lock where a tunnel catheter and subcutaneous PowerPort were attached for postoperative drug delivery. Two additional channels discontiguous with the drug delivery matrix were incorporated into the design for fixation with 2.7 mm cortical screws. After printing, the spacer was sterilized with STERRAD 100NX.

Comparative PMMA Cement Spacer Design and Preparation

Once the STTR spacer was constructed, a silicone mold was made using an OOMOO Silicone Mold Kit. The silicone mold was then utilized in the construction of a polymethylmethacrylate (PMMA) facsimile of the spacer utilizing Palacos R bone cement. Two grams of vancomycin powder were added to the cement before mixing. After drying for 24 hours, the PMMA spacer was weighed in order to calculate the relative value of antibiotic incorporated. The PMMA spacer was sterilized for surgical implantation in the same manner as the STTR spacer.

Implantation and Explantation

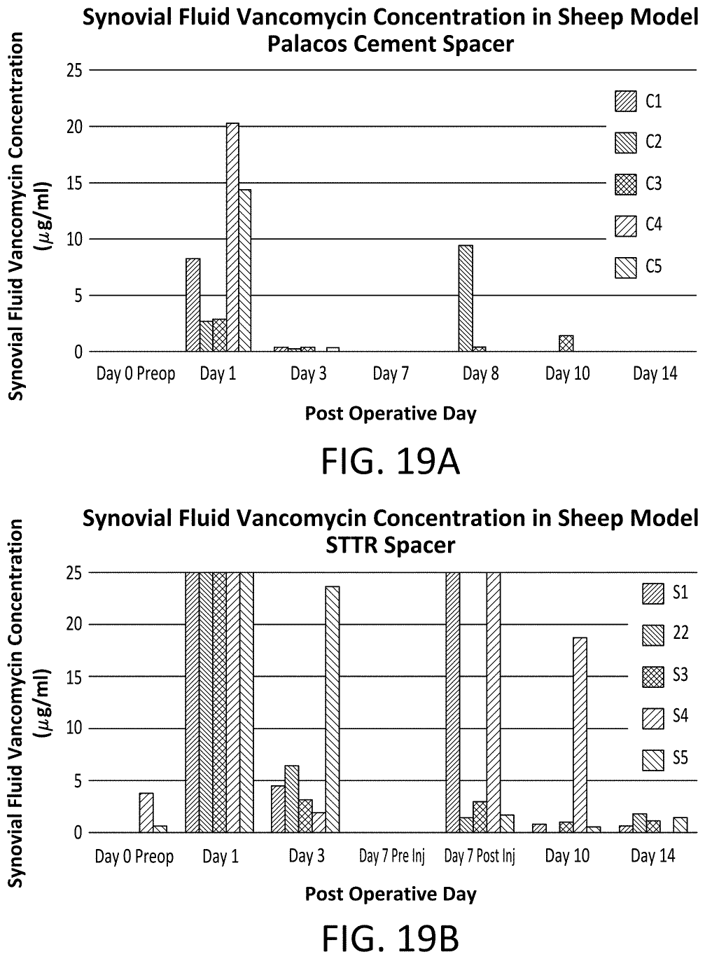

Seventeen (eight male, nine female) skeletally mature Dorset sheep underwent implant and explant of the prosthetic device in the right hindquarter over a two-week period. The animals were divided into four groups: five sheep received a vancomycin-impregnated PMMA spacer, five sheep received a STTR spacer with planned interval vancomycin dosing, five sheep received an STTR spacer with planned interval dosing of vancomycin along with anticoagulation protocol and 2 sheep received a STTR spacer with no antibiotic infusion. In all cases, a subfascial catheter was connected to either the STTR spacer or placed within the capsule of the PMMA spacer and then connected to the subcutaneous port with a 5 cc normal saline bolus given to confirm patency. FIG. 18A shows the surgical site immediately after implantation of an STTR knee spacer, with the spacer being implanted at site 1100 (the knee), and a subcutaneous port for drug injection at site 620A. FIG. 18B shows the postoperative sheep with an implanted STTR spacer and subcutaneous port.

Two weeks after implantation, the devices were explanted. On the day of explantation (Day 14), patency of the aperture tubing as well as the channels within the STTR spacer was confirmed by injecting Isovue 300 through the aperture and visualized fluoroscopically. FIG. 18C shows the surgical site with port 62 and affected site, i.e., the knee, exposed prior to explantation.

Sample Collection

For each animal, synovial fluid from the operative knee was collected both immediately before and after surgery on Day 0 as well as on Days 3, 7, 10 and on Day 14 just prior to explantation. Synovial fluid was analyzed for vancomycin concentration. Blood samples taken from the jugular vein were obtained at identical time points and analyzed for vancomycin concentration. Bone and synovium samples were taken on Day 0 just prior to implantation of either device and on Day 14 immediately after explantation and assessed for bacterial contamination and vancomycin concentration.

Vancomycin Concentration Determination

On the day of collection, blood samples were centrifuged at 2000×g for 10 minutes to separate serum from cells. The supernatant was extracted and stored at −80° C. Synovial fluid was similarly frozen and stored. The explanted PMMA spacer was placed in 750 mL sterilized phosphate buffered saline (PBS) in a 1000 mL beaker. After 48 hours on a hot plate set to 38° C. with a stir bar set at 150 rpm, the solution was homogenized, and a 10 mL sample was frozen at −80° C. for storage. For subsequent preparation for vancomycin concentration analysis, synovial fluid, serum, or PBS spacer solution was thawed and 50 μL of the sample and 50 μL of 0.25 μg/mL aminopterin solution were vortexed for 5 seconds. 200 μL of methanol were then added and centrifuged for 5 minutes at 15,000×g. 50 μL of the clear supernatant was then added to 500 μL of deionized water and analyzed for vancomycin concentration using mass spectrometry.

Culture Results

Using sterile technique, up to 0.5 g of either bone or synovium were crushed and placed in 5 mL of sterilized PBS and homogenized for 30 seconds. These samples were then placed at 4° C. overnight. For analysis, these samples were centrifuged for 10 minutes at 15,000×g. 50 μL of the supernatant and 50 μL of the 0.25 μg/mL aminopterin solution were vortexed for 5 seconds. 200 μL of methanol were then added and centrifuged for 5 minutes at 15,000×g. 50 μL of the clear supernatant was then analyzed for vancomycin concentration using mass spectrometry.

For bacterial culture, 400 μL samples of the thawed homogenized bone and synovium solutions were placed onto a blood agar plate and incubated at 37° C. for 24 hours and the colonies were counted. Plates with 0 colonies after 24 hours were put back in incubator at 37° C. for an additional 24 hours to ensure no growth.

Results

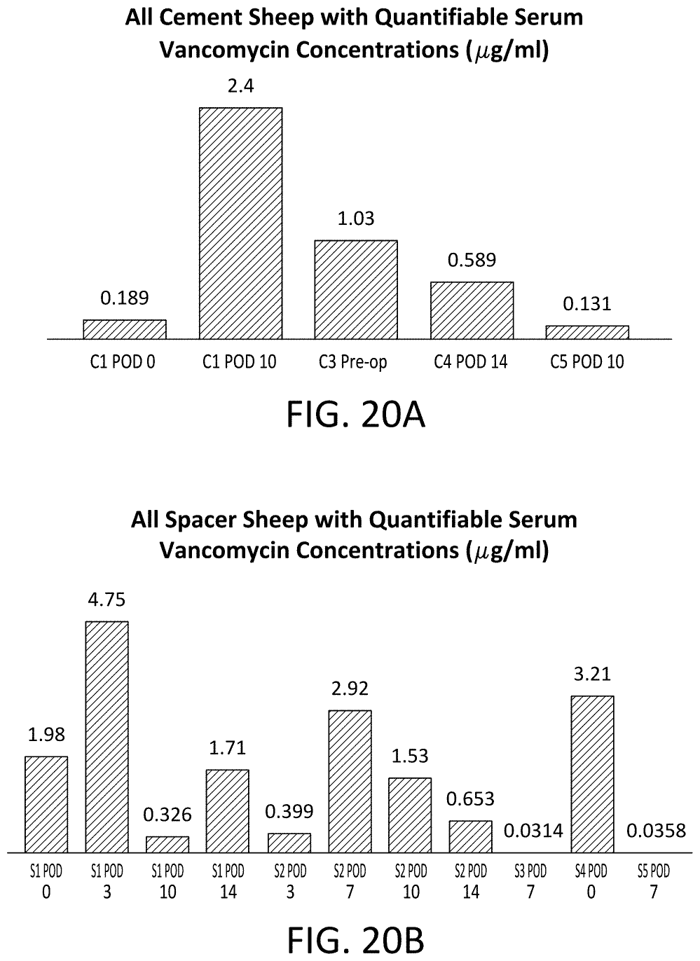

The PMMA spacers represented the standard of care and, as shown in FIG. 20A, did not produce vancomycin concentrations above 20 μg/ml, even on Day 1. Drug levels quickly decreased over time to low levels (<1 μg/ml). Drug levels necessary to treat free floating bacteria were determined to be 1-10 μg/ml. Drug levels necessary to treat biofilm infections were determined to be 10-100 μg/ml. Drug levels increased after redosing of antibiotics on Day 7 in Comparative Specimen number C2; however, specimen C2 did not produce vancomycin concentrations above 20 μg/ml, even after redosing on day 8. Other comparative specimens did not show a significant increase in vancomycin concentration on redosing. Thus, the PMMA bone cement spacers did not reliably combat infection.

As shown in FIG. 20B, concentrations for the STTR spacers increased above levels of quantification, i.e., >375 μg/ml, on Day 1. Drug levels increased after redosing of antibiotics on Day 7 in Specimen numbers S1 and S4. Additionally, in the STTR spacer group, even with elevated levels of vancomycin within the knee joint, levels within the blood never became toxic, as shown in FIG. 20B. In the comparative PMMA bone cement spacers, also had low levels of vancomycin within the blood, as shown in FIG. 20A

### Example 2

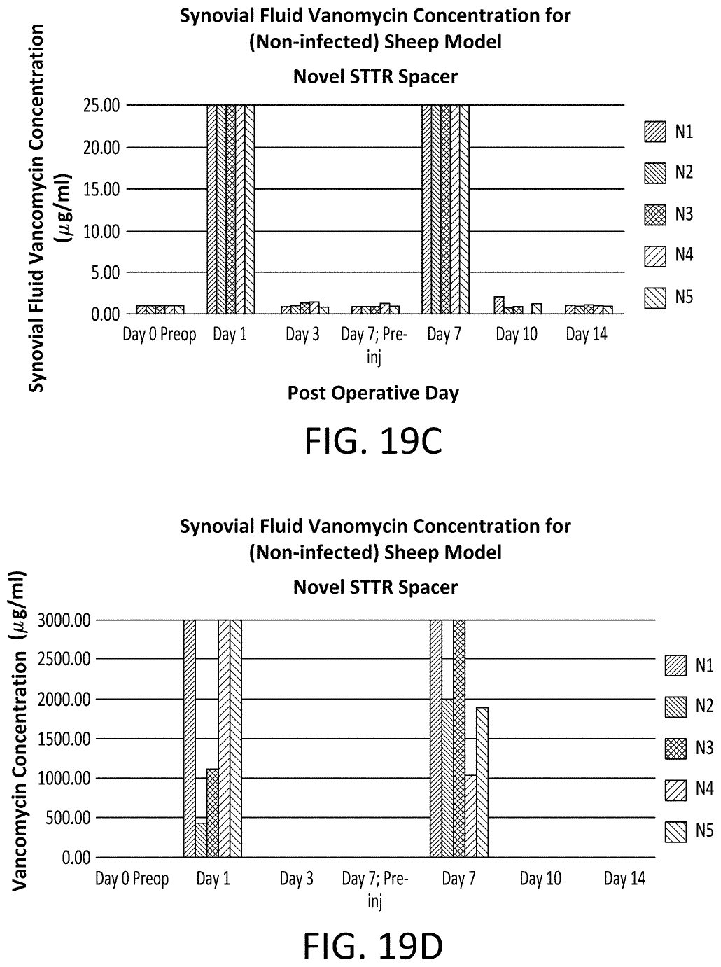

FIGS. 19C and 19D show results from a selective targeted treatment release (STTR) spacer 700 with channels 720 which were treated with an anticoagulant formulation to prevent clotting within a channel 720 or at the openings 721 at the surface of the spacer 720. In the spacer used in this example, the anticoagulant formulation is heparin in glycerol, although other anticoagulants known in the art may be used.

As discussed above for STTR spacers without an anticoagulant film, vancomycin concentrations released from the STTR spacers increased above 375 μg/ml, upon treatment with a vancomycin formulation on Day 1, as shown in FIG. 19B. Drug levels increased after redosing of antibiotics on Day 7 in to levels >375 μg/ml in Specimen numbers S1 and S4; however, some spacers, i.e., spacers S2, S3, and S5, showed little or no increase in drug released after redosing on day 7, also as shown in FIG. 18B.

FIGS. 19C and 19D, where 19C and 19D show similar results except that the scale on the y-axis is expanded in FIG. 19D, show vancomycin concentrations released from STTR spacers N1 to N5, manufactured in the same manner as the STTR spacers 700 of Example 1, except that the channels 720 are coated with a heparin/glycerin anticoagulant formulation. As shown in FIGS. 19C and 19D, concentrations of vancomycin in the tissue surrounding the STTR spacers increased to >375 μg/ml on Day 1, after administration of the drug through channels 720 of the spacer. Drug levels also increased to >375 μg/ml after redosing of vancomycin on Day 7 in each of the heparin-treated specimens N1 to N5. Thus, as shown in FIGS. 19C and 19D, treatment of channels within the STTR spacer with an anticoagulant formulation leads to improved drug release, even after the spacer is within a patient's body for an extended period. This is presumably due to a reduced likelihood of clotting within channels 720 and/or at openings 721 at the surface of the spacer 720.

### Example 3

### Cadaver Placement of STTR Knee Implant

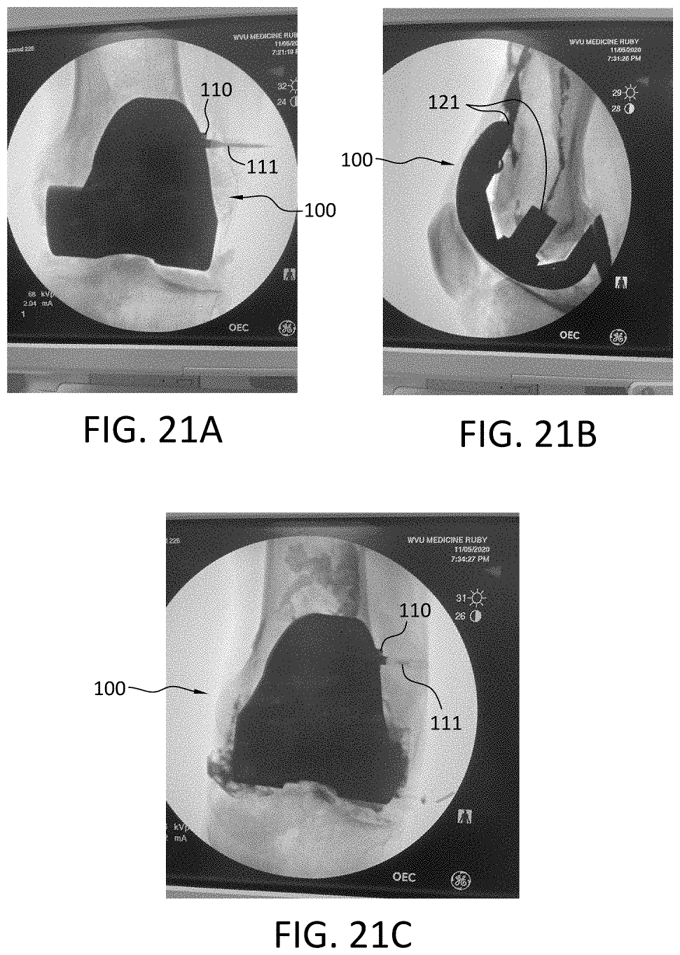

FIG. 21A shows a knee spacer 100 implanted in a cadaver, where spacer 100 includes an aperture 110 connected to tubing or catheter 111. A radiopaque dye, representing an antibiotic, is injected into a subcutaneous port 62 (not shown in FIG. 21A), in fluid communication with aperture 111, and the dye enters spacer 100 through aperture 110. The dye travels through channels 120 in spacer 100 and, as shown in FIG. 21B, the dye exits the spacer through openings 121 and enters affected tissue surrounding spacer 100. FIG. 21C shows radiopaque dye in the affected tissue surrounding spacer 100, after administration of the dye to the spacer.

### Example 4

### Cadaver Placement of STTR Hip Implant

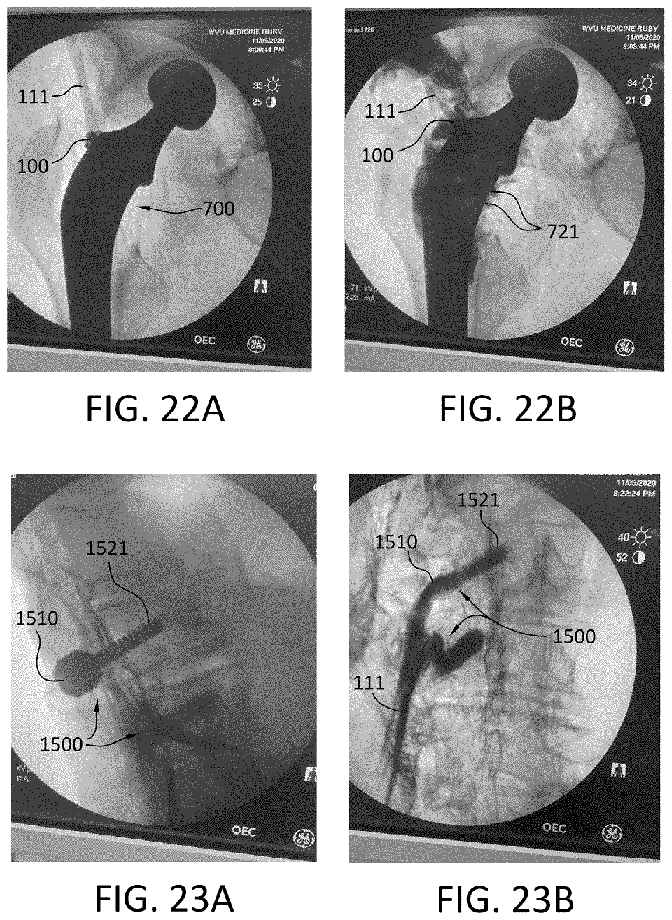

FIG. 22A shows a hip spacer 700 implanted in a cadaver, where spacer 700 includes an aperture 110 connected to tubing or catheter 111. A radiopaque dye, representing an antibiotic, is injected into a subcutaneous port 62 (not shown in FIG. 22A), in fluid communication with aperture 111, and the dye enters spacer 700 through aperture 110. The dye travels through channels 720 in spacer 700, and, as shown in FIG. 22B, then exits the spacer through openings 721 and enters affected tissue surrounding spacer 700.

### Example 5

### Cadaver Placement of STTR Pedicle Implants

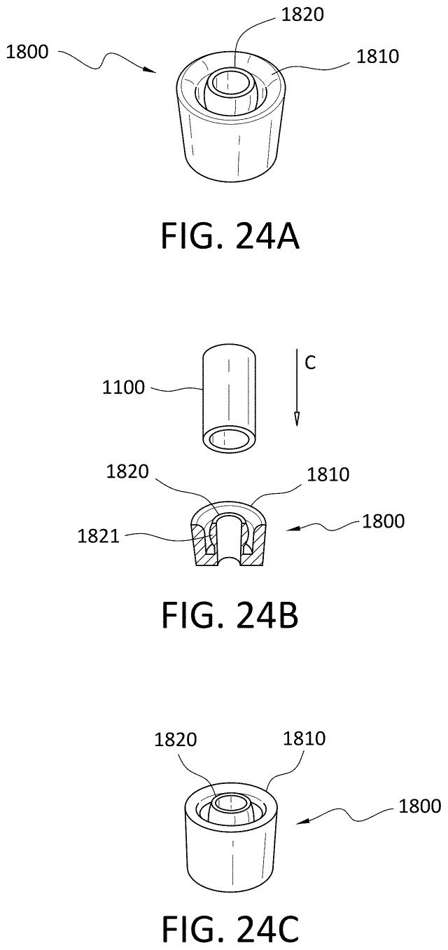

FIG. 23A shows two pedicle spacers 1500 implanted in adjacent vertebrae of a cadaver, where each spacer 1500 includes an aperture 1510, which may be connected to tubing or catheter 111. A radiopaque dye, representing an antibiotic, is injected into a subcutaneous port 62 (not shown), in fluid communication with aperture 111, and the dye enters spacer 1500 through aperture 1510, as seen in FIG. 23B. The dye travels through channels 1520 in spacer 1500, and, as shown in FIG. 23B, the dye exits the spacer through openings 1521 and enters affected tissue surrounding the pedicle spacers 1500.

### Aperture Design

Prior examples showed aperture 110 on a spacer 100 as a Luer lock type connection. Aperture 110 may be a male Luer lock fitting, configured to mate with a female Luer lock fitting on tubing 111. Luer lock style connectors are securely joined by means of a tabbed hub on the female fitting which screws into threads in a sleeve surrounding the male fitting. Luer slip connectors may also be used. Such Luer slip connectors are similar to Luer lock connectors, except that they lack a tab on the female fitting and a thread on the sleeve. Luer slip connectors are held together by friction.

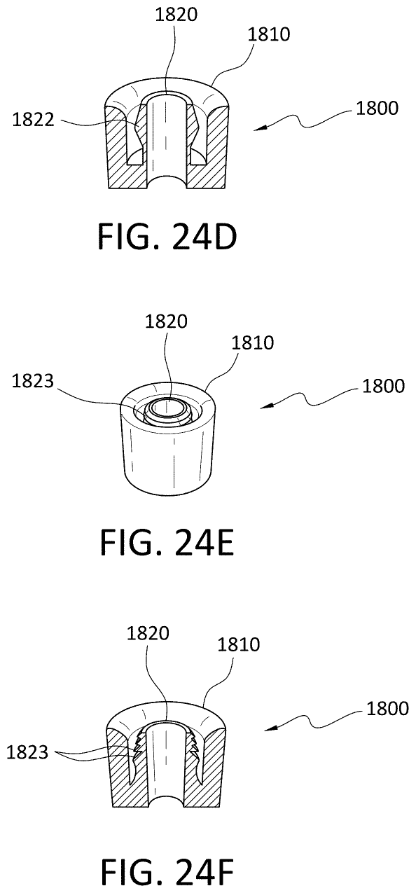

Additional ports 1800 which may be used as apertures on a spacer 100 are shown in FIGS. 24A to 24F. FIGS. 24A and 24B show perspective and cross section views of a port 1800 with an outer sleeve 1810 surrounding a tubular male joint 1820. A tube 111 may be connected to port 1800 by sliding the wall of tube 111 into the space between an inner surface of sleeve 1810 and an outer surface of male joint 1820 in the direction of arrow C. As seen in FIG. 24B, a bulbous surface 1821 is on the outer surface of male joint 1820. As tube 111 slides into the space between sleeve 1810 and male joint 1820, the wall of tube 111 is compressed between bulbous surface 1821 and sleeve 1810, holding tube 111 in position on port 1800.

A second embodiment of port 1800 is shown in FIGS. 24C and 24D. FIGS. 24C and 24D show a port 1800 with an outer sleeve 1810 surrounding a tubular male joint 1820, so that a tube 111 may be connected to port 1800 by sliding the wall of tube 111 into the space between an inner surface of sleeve 1810 and an outer surface of male joint 1820. As seen in FIG. 24D, the outer surface of male joint 1820 has a gradually increasing diameter, until a maximum diameter is reached at point 1822. The space between sleeve 1810 and male joint 1820 thus gradually decreases. As the wall of tube 111 enters the space between sleeve 1810 and male joint 1820, it becomes increasingly compressed between bulbous surface 1821 and sleeve 1810, holding tube 111 in position on port 1800. In various embodiments, tube 111 is held in position between sleeve 1810 and male joint 1820 at point 1822.

A third embodiment of port 1800 is shown in FIGS. 24E and 24F. FIGS. 24E and 24F show a port 1800 with an outer sleeve 1810 surrounding a tubular male joint 1820, so that a tube 111 may be connected to port 1800 by sliding the wall of tube 111 into the space between an inner surface of sleeve 1810 and an outer surface of male joint 1820. As seen in FIGS. 24E and 24F, the outer surface of male joint 1820 has a series of downwardly directed angled flanges 1823. As the wall of tube 111 enters the space between sleeve 1810 and male joint 1820, it passes downwards past flanges 1823. In various embodiments, flanges 1823 hold tube 111 in position between sleeve 1810 and male joint 1820. Tube 111 is not easily removed, because flanges 1823 do not readily bend upwards to allow disconnection of tube 111.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A system to replace or substitute a medical implant in a patient comprising:
a housing comprising a shape similar to the medical implant;
an aperture;
a subcutaneous port configured to receive a medicament, the subcutaneous port being in fluid communication with the aperture; and
at least one channel within the housing, wherein the at least one channel extends from the aperture to an exterior surface of the housing to equally distribute the medicament from the aperture through the at least one channel,
wherein the subcutaneous port is in fluid communication with the aperture through a catheter connecting the subcutaneous port to the aperture.

2. The system of claim 1, wherein the at least one channel extends to an exterior surface of the housing at a particular location chosen to treat an infection, bone loss, or tumors.

3. The system spacer of claim 1, wherein the housing comprises a shape similar to the medical implant, wherein the medical implant is a hip implant, an intramedullary nail, a pedicle screw, a knee implant, a sternum prosthesis, a clavicle prosthesis, an ankle implant, a shoulder implant, a tibia implant, a femur implant, a humerus implant, a spinal cage, an external fixation pin, an intercalary fusion device, a talus implant, a vertebral body implant, or a bone replacement.

4. The system of claim 1, wherein the at least one channel comprises an oval, circular, square, irregular, triangular, or hexagonal cross-section.

5. The system of claim 1, wherein the material is equally distributed to an infection site of a patient.

6. The system of claim 4, wherein the infection site is cancellous or cortical bone or a soft tissue site.

7. The system of claim 1, wherein the material is a treatment agent.

8. The system of claim 7, wherein the treatment agent is selected from the group consisting of an antibiotic, an antifungal, a bacteriophage fluid, an antimicrobial peptides, a surfactant, an enzyme, a wash, an antiseptic, pain medication, a chemotherapeutic agent, an immunotherapeutic agent, an anti-thrombotic agent, and an anti-inflammatory.

9. The system of claim 8, wherein the treatment agent is an antibiotic.

10. The system of claim 1, wherein the at least one channel has a diameter of between 100 to 2000 microns.

11. The system of claim 1, wherein the at least one channel has a varying diameter.

12. A method of manufacturing a spacer comprising printing a solvent-soluble channel pattern for use in a pre-existing spacer mold.

13. The method of claim 12, wherein the solvent-soluble channel pattern is printed using additive manufacturing techniques.

14. The method of claim 13, wherein the additive manufacturing technique is 3D printing.

15. The method of claim 12, wherein the solvent-soluble channel pattern is printed from a water-soluble resin.

16. The method of claim 12, wherein the solvent-soluble channel pattern is printed from an organic solvent-soluble resin.

17. A method of manufacturing a spacer to replace or substitute for a medical implant, said spacer comprising: a housing comprising a shape similar to the medical implant; an aperture; and at least one channel within the housing, wherein the at least one channel extends from the aperture to an exterior surface of the housing, the method comprising:
printing the spacer by additive manufacturing, wherein the spacer has the same shape as the medical implant replaced by the spacer.

18. The method of claim 17, wherein the additive manufacturing technique is 3D printing.

19. The method of claim 17, wherein the at least one channel comprises an oval, circular, triangular, square, irregular, or hexagonal cross-section.

20. The method of claim 17, wherein the at least one channel has a diameter of between 100 to 2000 microns.

21. The method of claim 17, wherein the at least one channel has a varying diameter.

22. A preform for a spacer to replace a medical implant in a patient, the preform comprising:
a housing comprising a shape similar to the medical implant;
an aperture; and
a 3D-printed solvent-soluble network pattern within the housing, wherein the 3D-printed network pattern extends from the aperture to a plurality of openings on an exterior surface of the housing,
wherein:
the solvent-soluble network pattern is configured to be dissolved from the housing so as to form channels in the medical implant, wherein said channels place the aperture in fluid communication with the plurality of openings.

23. The preform of claim 22, wherein the solvent-soluble network pattern is a water-soluble network pattern.

24. The spacer of claim 22, wherein the solvent-soluble network pattern is an organic solvent-soluble network pattern, wherein the organic solvent-soluble network pattern is made from polycaprolactone or HIPS.

25. The preform of claim 22, wherein the housing comprises a shape similar to a medical implant selected from the group consisting of a hip implant, an intramedullary nail, a pedicle screw, a knee implant, a sternum prosthesis, a clavicle prosthesis, an ankle implant, a shoulder implant, a tibia implant, a femur implant, a humerus implant, a spinal cage, an external fixation pin, an intercalary fusion device, a talus implant, a vertebral body implant, or a bone replacement.

26. A spacer to replace a medical implant in a patient, comprising:
a housing comprising a shape similar to the medical implant;
an aperture; and
a network of channels within the housing, wherein the network of channels extends from the aperture to a plurality of openings on an exterior surface of the housing,
wherein:
the spacer is prepared from the preform of claim 21 by dissolving the 3D-printed solvent-soluble network pattern from the housing of the preform so as to form the network of channels, wherein the network of channels places the aperture in fluid communication with the plurality of openings.

27. A method for replacing a medical implant in a patient, where the implant is at a site in need of treatment, comprising:
removing the medical implant from the patient;
replacing the medical implant with a spacer comprising a housing comprising a shape similar to the medical implant; an aperture; and at least one channel within the housing, wherein the at least one channel extends from the aperture to an exterior surface of the housing;
implanting a subcutaneous port configured to receive a medicament in the patient, and placing the subcutaneous port in fluid communication with the aperture through a catheter connecting the subcutaneous port to the aperture.

28. The method of claim 27, further comprising a step of supplying the medicament to the subcutaneous port in an amount sufficient to cause the medicament to travel through the catheter to the at least one channel in the housing.

29. The method of claim 27, further comprising a step of supplying an anticoagulant to the subcutaneous port in an amount sufficient to cause the anticoagulant to coat the at least one channel in the housing.

30. The method of claim 27, wherein the site in need of treatment is a site of infection, a site undergoing bone loss, or a site of a tumor.
